(19) United States
(12) Patent Application Publication
(10) Pub. No.: US 2025/0115624 A1
(43) Pub. Date: Apr. 10, 2025

(54) BORON COMPOUNDS, RELATED LIPID PARTICLES, LIPOPLEXES, COMPOSITIONS, METHODS OF USE, AND METHODS OF MAKING

(71) Applicant: UNIVERSITY OF LOUISVILLE RESEARCH FOUNDATION, INC., Louisville, KY (US)

(72) Inventors: Michael H. NANTZ, Louisville, KY (US); Faisal IBRAHIM, Paterson, NJ (US); Prasadanie K. ADHIHETTY, Louisville, KY (US)

(73) Assignee: UNIVERSITY OF LOUISVILLE RESEARCH FOUNDATION, INC., Louisville, KY (US)

(21) Appl. No.: 18/858,318

(22) PCT Filed: Apr. 27, 2023
(86) PCT No.: PCT/US2023/066271

§ 371 Date: Oct. 20, 2024

Related U.S. Application Data
(60) Provisional application No. 63/363,716, filed on Apr. 28, 2022.

(51) Int. Cl. C07F 5/02; A61K 9/127; A61K 31/69

(52) U.S. Cl. CPC C07F 5/027; A61K 9/127; A61K 31/69

(57) ABSTRACT

Some embodiments of the invention include inventive compounds (e.g., boron compounds of Formula (I)), inventive lipid particles, and inventive lipoplexes. Other embodiments include compositions (e.g., pharmaceutical compositions) comprising the inventive compound, inventive lipid particles, or inventive lipoplexes. Some embodiments include methods of using the inventive lipoplexes (e.g., in compositions or in pharmaceutical compositions) for administering, treating disease, inducing an immune response, and combinations thereof. Further embodiments include methods for making the inventive compounds. Additional embodiments of the invention are also discussed herein.

### CROSS-REFERENCE TO RELATED APPLICATIONS

This application claims the benefit of U.S. Provisional Application No. 63/363,716, filed Apr. 28, 2022, entitled “BORON COMPOUNDS, RELATED LIPID PARTICLES, COMPOSITIONS, AND METHODS OF USE” which is herein incorporated by reference in its entirety.

### REFERENCE TO A SEQUENCE LISTING

The instant application contains a Sequence Listing that has been submitted in XML format via EFS-Web and is hereby incorporated by reference in its entirety. Said XML copy, created on Apr. 24, 2023, is named 2023_04_26_35783_04194_PCT.xml and is 4 KB in size.

### BACKGROUND

Delivery of nucleic acids to cells or animals can sometimes be difficult, and in some instances can prevent the treatment of disease. To date, methods to deliver nucleic acids are often limited and sometimes inefficient. Some embodiments of the present invention include boron compounds and their uses (e.g., in lipoplexes) to deliver nucleic acids. Additional embodiments of the invention are also discussed herein.

### SUMMARY

Some embodiments of the invention include inventive compounds (e.g., boron compounds of Formula (I)), inventive lipid particles, and inventive lipoplexes. Other embodiments include compositions (e.g., pharmaceutical compositions) comprising the inventive compound, inventive lipid particles, or inventive lipoplexes. Some embodiments include methods of using the inventive lipoplexes (e.g., in compositions or in pharmaceutical compositions) for administering, treating disease, inducing an immune response, and combinations thereof. Further embodiments include methods for making the inventive compounds. Additional embodiments of the invention are also discussed herein.

Some embodiments of the invention include boron compounds selected from Formula (I), salts of Formula (I), optical isomers of Formula (I), geometric isomers of Formula (I), salts of optical isomers of Formula (I), salts of geometric isomers of Formula (I), and derivatives thereof, where Formula (I) is Formula (Ia) and Formula (Ib),

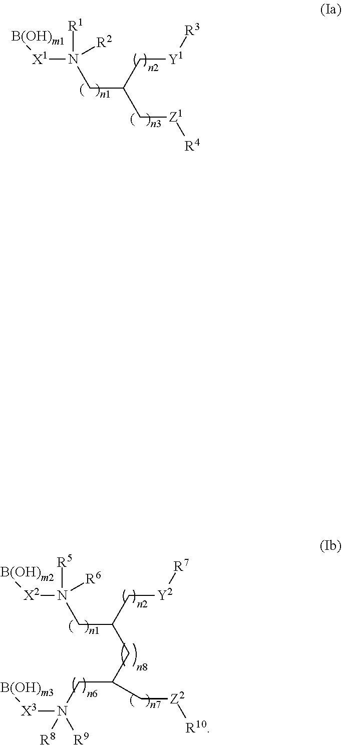

In certain embodiments, X¹, X², and X³ can be the same or different and are —C(R^{a})(R^{b})—, —C(R^{a})(R^{b})—C(R^{c})(R^{d})—, or —C(R^{a})(R^{b})—C(R^{c})(R^{d})—C(R^{e})(R^{f})—, where R^{a}, R^{b}, R^{c}, R^{d}, R^{e}, and R^{f} can be the same or different if they are on different X groups. In other embodiments, R^{a}, R^{b}, R^{c}, R^{d}, R^{e}, and R^{f} can be the same or different and are H, halogen (e.g., F, Cl, Br, or I), —CN, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, or —OCF₃. In some embodiments, R¹, R², R⁵, R⁶, R⁸, and R⁹ can be the same or different and are a lone pair of electrons, H, hydroxy (—OH), methanoyl (—COH), nitro (—NO₂), —NH₂, —N(CH₃)₂, cyano (—CN), sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, —OCF₃, C₁-C₁₀ alkyl, C₂-C₁₀ alkenyl, C₂-C₁₀ alkynyl, or C₁-C₉ alkoxy, which methanoyl (—COH), —NH₂, —N(CH₃)₂, —CONH₂, —CON(CH₃)₂, —C(O)(CH₃), —C(O)(C₂H₅), —C(O)(C₃H₇), C₁-C₁₀ alkyl, C₂-C₁₀ alkenyl, C₂-C₁₀ alkynyl, C₁-C₉ alkoxy can optionally be substituted with one or more of halogen, oxo (=O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluoronated alkyl, —CF₃, —OCF₃, or C₁-C₃ alkoxy. In still other embodiments, m1 is 2 or 3. In yet other embodiments, m2 is 2 or 3. In certain embodiments, m3 is 2 or 3. In other embodiments, n1 is 1, 2, 3, 4, 5, 6, 7, 8, or 9. In some embodiments, n2 is 0, 1, 2, 3, or 4. In still other embodiments, n3 is 0, 1, 2, 3, or 4. In yet other embodiments, n4 is 1, 2, 3, 4, 5, 6, 7, 8, or 9. In certain embodiments, n5 is 0, 1, 2, 3, or 4. In some embodiments, n6 is 0, 1, 2, 3, 4, 5, 6, 7, 8, or 9. In other embodiments, n7 is 0, 1, 2, 3, or 4. In some embodiments, n8 is 0, 1, 2, or 3. In still other embodiments, Y¹ and Y² can be the same or different and are —CH₂—, —O—(CO)—, —(CO)—O—, —O—(N═CH)—, —(CH═N)—O—, —O—, —N(R^{g})—(CO)—, or —(CO)—N(R^{g})—, where R^{g} can be the same or different if they are on different Y groups. In yet other embodiments, Z¹ and Z² can be the same or different and are —CH₂—, —O—(CO)—, —(CO)—O—, —O—(N═CH)—, —(CH═N)—O—, —O—, —N(R^{h})—(CO)—, or —(CO)—N(R^{h})—, where R^{h} can be the same or different if they are on different Z groups. In certain embodiments, R⁹ and R^{h} can be the same or different and are H, methyl, ethyl, propyl, or butyl. In other embodiments, R³, R⁴, R⁷ and R¹⁰ can be the same or different and are C₆-C₃₀ alkyl, C₆-C₃₀ alkenyl, or C₆-C₃₀ alkynyl.

In certain embodiments, Formula (I) is

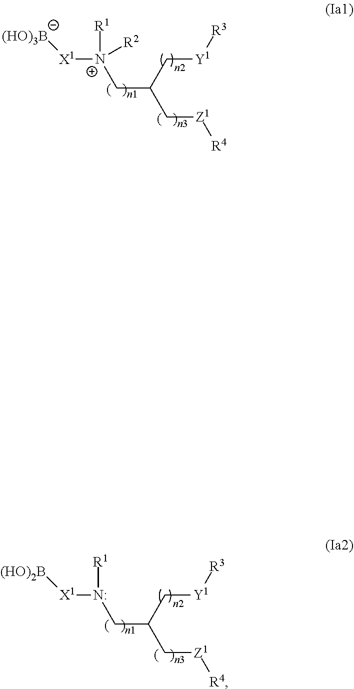

where R¹ is not a lone pair of electrons in Formula (Ia2). In some embodiments, X¹, X², and X³ is —C(R^{a})(R^{b})—. In other embodiments, R^{a}, R^{b}, R^{c}, R^{d}, R^{e}, and R^{f} are H. In yet other embodiments, R¹ and R² can be the same or different and are H, methyl, or ethyl. In still other embodiments, R⁵ and R⁶ can be the same or different and are H, methyl, or ethyl. In certain embodiments, R⁸ and R⁹ can be the same or different and are H, methyl, or ethyl. In other embodiments, R³ and R⁴ are the same or different and are C₁₂-C₁₈ alkyl or C₁₂-C₁₈ alkenyl. In some embodiments, R⁷ and R¹⁰ are the same or different and are C₁₂-C₁₈ alkyl or C₁₂-C₁₈ alkenyl. In still other embodiments, Y¹ and Y² are the same or different and are —O—(CO)—, —O—(N═CH)—, or —O—. In certain embodiments, Z¹ and Z² are the same or different and are —O—(CO)—, —O—(N═CH)—, or —O—. In certain embodiments, X¹ is —CH₂—, R¹ is —CH₃, R² is —CH₃, n1 is 1, n2 is 0, n3 is 1, Y¹ is —O—(CO)—, or Z¹ is —O—(CO)—, or a combination thereof. In other embodiments, X² is —CH₂—, X³ is —CH₂—, R⁵ is —CH₃, R⁶ is —CH₃, R⁸ is —CH₃, R⁹ is —CH₃, n4 is 1, n6 is 0, n5 is 0, n7 is 0, n8 is 0, Y² is —O—(CO)—, or Z² is —O—(CO)—, or a combination thereof. In some embodiments, a pKₐ of the boron compound is 5 to 8 or 6 to 7. In yet other embodiments, the boron compound is I-1, I-2, I-3, I-4, I-5, I-6, I-7, I-8, I-9, I-10, I-11, I-12, I-13, I-14, I-15, I-16, or I-17. In still other embodiments, the boron compound is I-1, I-2, or I-3.

Certain embodiments of the invention include lipid particles comprising the boron compound (e.g., as described herein). In certain embodiments, the amount of the boron compound in the lipid particle is 0.01 to 50 mol %. In other embodiments, (a) the boron compound reacts with a lipid particle component in the lipid particle to form one or more covalent bonds between the boron compound and the lipid particle component, and (b) the lipid particle component is not a boron compound. In some embodiments, the lipid particle component is a molecule with a diol, a molecule with a 1,2 diol, a molecule with a 1,3 diol, or OEL4. In still other embodiments, the diameter of the lipid particle is decreased by at least 10%, compared to the lipid particle without addition of the boron compound. In some embodiments, the diameter of the lipid particle is 30-250 nm.

Some embodiments of the invention include lipoplexes comprising (a) the boron compound (e.g., as described herein) or the lipid particle (e.g., as described herein) and (b) a nucleic acid molecule. In certain embodiments, the nucleic acid molecule has a molecular weight (in daltons) of no more than 10,000,000.

Some embodiments of the invention include compositions comprising the boron compound (e.g., as described herein), the lipid particle (e.g., as described herein), or the lipoplex (e.g., as described herein).

Some embodiments of the invention include pharmaceutical compositions comprising (a) the boron compound (e.g., as described herein), the lipid particle (e.g., as described herein), or the lipoplex (e.g., as described herein) and (b) optionally a formulary ingredient.

Some embodiments of the invention include methods for providing nucleic acid to a cell (e.g., plant, cell, animal cell, mammalian cell, or human cell) comprising one or more administrations to the cell of one or more compositions comprising the lipoplex (e.g., as described herein), the composition (e.g., as described herein), or the pharmaceutical composition (e.g., as described herein), wherein the compositions may be the same or different if there is more than one administration. In certain embodiments, the method silences a gene in the cell or the added nucleic acid results in the expression of a protein or a polypeptide in the cell. In other embodiments, the cell is in vivo, ex vivo, or in vitro. In some embodiments, the method induces an immune response.

Some embodiments of the invention include methods for providing an animal with a compound comprising one or more administrations to the animal of one or more compositions comprising the lipoplex (e.g., as described herein), the composition (e.g., as described herein), or the pharmaceutical composition (e.g., as described herein), wherein the compositions may be the same or different if there is more than one administration. In certain embodiments, at least one of the one or more compositions further comprises a formulary ingredient. In other embodiments, at least one of the one or more compositions comprises the composition (e.g., as described herein), or the pharmaceutical composition (e.g., as described herein). In some embodiments, at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration. In still other embodiments, if there is more than one administration at least one composition used for at least one administration is different from the composition of at least one other administration. In yet other embodiments, the lipoplex of at least one of the one or more compositions is administered to the animal in an amount of from about 0.01 mg/kg animal body weight to about 15 mg/kg animal body weight. In certain embodiments, the animal is a human, a rodent, or a primate. In some embodiments, the method induces an immune response.

Some embodiments of the invention include methods for treating an animal for a disease, comprising one or more administrations of one or more compositions comprising the lipoplex (e.g., as described herein), the composition (e.g., as described herein), or the pharmaceutical composition (e.g., as described herein), wherein the compositions may be the same or different if there is more than one administration. In certain embodiments, at least one of the one or more compositions further comprises a formulary ingredient. In other embodiments, at least one of the one or more compositions comprises the composition (e.g., as described herein) or the pharmaceutical composition (e.g., as described herein). In certain embodiments, at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration. In some embodiments, if there is more than one administration at least one composition used for at least one administration is different from the composition of at least one other administration. In still other embodiments, the lipoplex of at least one of the one or more compositions is administered to the animal in an amount of from about 0.005 mg/kg animal body weight to about 50 mg/kg animal body weight. In still other embodiments, the animal is a human, a rodent, or a primate. In certain embodiments, the animal is in need of the treatment. In other embodiments, the method is for treating cancer. In still other embodiments, the method is for treating acute lymphoblastic leukemia, astrocytoma, basal cell carcinoma, bladder cancer, bone marrow cancer, breast cancer, chronic lymphocytic leukemia (CLL), CNS cancer, colon cancer, colorectal cancer, endometrial cancer, gastric cancer, glioblastoma, glioblastoma multiforme, glioma, gliosarcoma, hepatocellular carcinoma, kidney cancer, leukemia, liver cancer, lung cancer, lymphoma, melanoma, malignant nerve sheath tumors, medulloblastoma, meningioma, multiple myeloma, nasopharyngeal carcinoma, neuroblastoma, non-Hodgkin lymphoma, non-small cell lung cancer, oral cancer, ovarian cancer, pancreatic cancer, prostate cancer, rectal cancer, renal cancer, renal cell carcinoma, rhabdomyosarcoma, squamous cell carcinoma, stomach cancer, thyroid cancer, uterine cancer, cancers that can result in metastasis, cancers resulting from metastasis, or cancerous tumors thereof. In some embodiments, the method is for treating cancerous tumors. In certain embodiments, the method comprises a vaccination. In other embodiments, the method is for treating infections. In still other embodiments, the method is for treating bacterial infections, viral infections, fungal infections, or a combination thereof. In some embodiments, the method induces an immune response.

Some embodiments of the invention include methods for preparing the boron compound of Formula (Ia) and salts thereof (e.g., as disclosed herein), comprising:
(a) reacting a compound of Formula (IIa) with R³—(O)C-(halogen) and R⁴—(O)C-(halogen) to result in a mixture comprising a compound of Formula (IIIa);
(b) reacting a compound of Formula (IIIa) with 2-(halomethyl)-4,4,5,5-tetramethyl-1,3,2-dioxaborolane to result in a mixture comprising a compound of Formula (IVa);
(c) reacting a compound of Formula (IV a) with any suitable molecule to oxidatively create B—OH groups; and
(d) recovering Formula (Ia) or a salt thereof,
where Formula (IIa) is

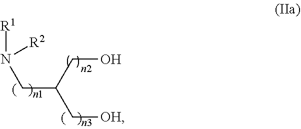

Formula (IIIa) is

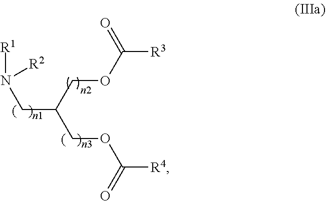

and

Formula (IVa) is

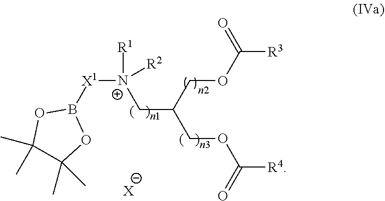

Some embodiments of the invention include methods for preparing the boron compound of Formula (Ib) and salts thereof (e.g., as disclosed herein), comprising:
(a) reacting a compound of Formula (IIb) with R⁷—(O)C-(halogen) and R¹⁰—(O)C-(halogen) to result in a mixture comprising a compound of Formula (IIIb);
(b) reacting a compound of Formula (IIIb) with 2-(halomethyl)-4,4,5,5-tetramethyl-1,3,2-dioxaborolane pinacol ester to result in a mixture comprising a compound of Formula (IVb);
(c) reacting a compound of Formula (IVb) with any suitable molecule to oxidatively create B—OH groups; and
(d) recovering Formula (Ib) or a salt thereof
where Formula (IIb) is

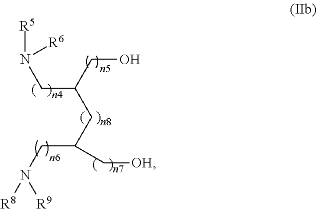

Formula (IIb) is

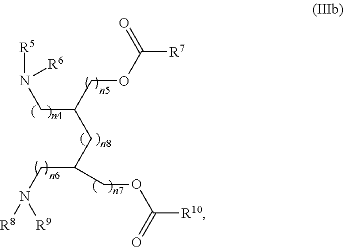

and
Formula (IVb) is

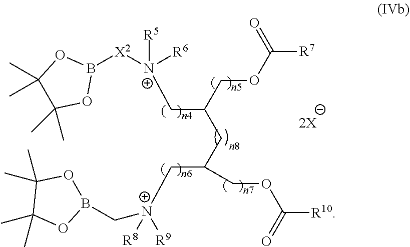

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Examples of synthesis of boron-containing lipids. The conditions of the reactions are: a. myristoyl chloride or oleoyl chloride, triethylainine (TEA), 4-(N,N-dimethylamino)pyridine (DMAP), CH₂Cl₂, 0° C., rt, 12 h (97%); b. 2-(bromomethyl)-4,4,5,5-tetramethyl-1,3,2-dioxaborolane, CH₂Cl₂, 50° C., sealed tube, 8 h (89%); c. NaOI₄, HCl (2.0 M), H₂O/THF 0° C.-rt, 12 h (71%).

FIG. 2: Role of sodium periodate in the boronic ester deprotection step.

FIG. 3: Gene silencing activities of OEL4-DsiRNA lipoplexes with and without DMDBH. OEL4:DOPE:DMDBH:DSPE-350 (1:1:0.01 to 0.10:0.03) liposomes were prepared and then mixed with anti-eGFP DsiRNA. The % eGFP silencing was determined relative to control cells (GFP-expressing MDA-MB-231). L2K is control experiment using industry standard transfection lipid Lipofectamine 2000.

FIG. 4: Gene silencing activities of OEL4-DMDBH (1:1)-DsiRNA lipoplexes formed via a pre-mix formulation protocol. DMDBH:DOPE (1:1) liposomes were mixed with anti-eGFP DsiRNA and incubated 30 mins before addition of OEL4:DOPE:DSPE-350 (1:1:0.01 to 0.05) liposomes to yield lipoplexes that then were added to GFP-expressing MDA-MB-231 cells.

FIG. 5: Gene silencing activity of OEL4-PBA (1:1)-DsiRNA lipoplexes formed via the pre-mix formulation protocol (PBA=phenylboronic acid). PBA:DOPE (1:1) was mixed with anti-eGFP DsiRNA and incubated 30 mins before addition of OEL4:DOPE:DSPE-350(1:1:0.03) liposomes to yield a lipoplex that then was added to GFP-expressing MDA-MB-231 cells. OEL4:DMDBH:DOPE:DSPE-350(1:1:2:0.03) lipoplexes were also prepared using the pre-mix formulation protocol and tested for comparison.

FIG. 6: Boronate ester formation produces BE₁M₄, a lipid with a (C₁₄)₄-hydrophobic domain. BE₁M₄=(#boronate ester links)₁(#myristyl chains)₄.

FIG. 7: Representation of liposome bilayer stabilization through formation of hydroxyboronate ester linkages (lines connecting lipid heads). Example shows a 1:1:1 DMDBH (dotted), OEL4 (open), and DOPE (splotched) lipid mixture.

FIG. 8: Boronic acid equilibria with diols to form boronate esters.

FIG. 9: Boronic acid pKₐ values.

FIG. 10: Agarose gel (2%) binding experiment showing binding of PEGylated liposomes (500 ng of lipid mixture) to Alexa-546-labelled RNA duplexes (25 ng per sample).

FIG. 11: Synthetic route for synthesis of bis(ammonium boronic acid) lipid 5. Reagents and conditions: a. 40% dimethylamine in H₂O, 0° C. to rt, 48 h; b. 2,4-dimethylpyridine, myristoyl chloride, triethylamine, CH₂Cl₂, 0° C. to rt, 18 h (83%); c. 2-(bromomethyl)-4,4,5,5-tetramethyl-1,3,2-dioxaborolane pinacol ester, CH₂Cl₂, 50° C. 24 h (50%); d. 0.1 M HCl in MeOH, methyl boronic acid, rt, 24 h (90%).

FIG. 12: Representation of reversible boronate ester crosslinking (lines connecting lipid heads) in a lipoplex bilayer leaflet when bis(boronic acid) DMTBH reacts with adjacent OEL4 lipids to form both mono- and bis-linked products. Key: DMTBH (dotted), OEL4 (open), and DOPE (splotched).

FIG. 13: Gene silencing activities of transfection lipid-DsiRNA liposome formulations with and without DMDBH. Liposomes were prepared and then mixed with anti-eGFP DsiRNA. The % eGFP silencing was determined relative to control cells (GFP-expressing MDA-MB-231 cells).

FIG. 14: 24-Well plate study design. Number of cells plated on 24-well plate: ca. 60,000 per well. Cells were plated overnight prior to the day of experiment. In this FIG. 12 table, “PEG350(3)” is 3 mol % of 1,2-distearoyl-sn-glycero-3-phosphoethanolamine-N-[methoxy(polyethylene glycol)-350](ammonium salt) was added to each liposome formulation. “iOEL4” and “eiOEL4” liposomes were prepared in pH 4 acetate buffer. “OEL4” and “DMDBH” liposomes were prepared in pH 7 nuclease free water. All liposomes contain equimolar amount of DOPE relative to the transfection lipid or DMDBH. “L2K” is Lipofectamine-2000.

FIG. 15: Transfection activity of OEL4:DOPE:DSPE-PEG350 (1:1:0.03) liposomes without and with equimolar boron lipid DMDBH at different Luc mRNA doses.

FIG. 16: Cell viability assay to accompany the transfection experiments depicted in FIG. 15.

FIG. 17: Transfection activity of OEL/Luc mRNA formulations relative to standard lipid formulation SM-102 in HeLa cells.

FIG. 18: Influence of mRNA dose (25-100 ng/well) on transfection activity of LNP/Luc mRNA complexes.

### DETAILED DESCRIPTION

Some embodiments of the invention include inventive compounds (e.g., boron compounds of Formula (I)), inventive lipid particles, and inventive lipoplexes. Other embodiments include compositions (e.g., pharmaceutical compositions) comprising the inventive compound, inventive lipid particles, or inventive lipoplexes. Some embodiments include methods of using the inventive lipoplexes (e.g., in compositions or in pharmaceutical compositions) for administering, treating disease, inducing an immune response, and combinations thereof. Further embodiments include methods for making the inventive compounds. Additional embodiments of the invention are also discussed herein.

As used herein (unless otherwise specified), the term “alkyl” means a monovalent, straight or branched hydrocarbon chain. For example, the terms “C₁-C₇ alkyl” or “C₁-C₄ alkyl” refer to straight- or branched-chain saturated hydrocarbon groups having from 1 to 7 (e.g., 1, 2, 3, 4, 5, 6, or 7), or 1 to 4 (e.g., 1, 2, 3, or 4), carbon atoms, respectively. Examples of C₁-C₇ alkyl groups include, but are not limited to, methyl, ethyl, n-propyl, i-propyl, n-butyl, s-butyl, t-butyl, n-pentyl, s-pentyl, n-hexyl, and n-septyl. Examples of C₁-C₄ alkyl groups include, but are not limited to, methyl, ethyl, n-propyl, i-propyl, n-butyl, s-butyl, and t-butyl.

As used herein (unless otherwise specified), the term “alkenyl” means a monovalent, straight or branched hydrocarbon chain that includes one or more (e.g., 1, 2, 3, or 4) double bonds. Examples of alkenyl groups include, but are not limited to, vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

As used herein (unless otherwise specified), the term “alkoxy” means any of the above alkyl groups which is attached to the remainder of the molecule by an oxygen atom (alkyl-O—). Examples of alkoxy groups include, but are not limited to, methoxy (sometimes shown as MeO—), ethoxy, isopropoxy, propoxy, and butyloxy.

As used herein (unless otherwise specified), the term “alkynyl” means a monovalent, straight or branched hydrocarbon chain that includes one or more (e.g., 1, 2, 3, or 4) triple bonds and that also may optionally include one or more (e.g. 1, 2, 3, or 4) double bonds in the chain. Examples of alkynyl groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl.

As used herein (unless otherwise specified), the term “aryl” means a monovalent, monocyclic or bicyclic, 5, 6, 7, 8, 9, 10, 11, or 12 membered aromatic hydrocarbon group which, when unsubstituted. Examples of aryl groups include, but are not limited to, phenyl, naphthyl, tolyl, and xylyl. For a bicyclic aryl that is designated as substituted, one or both rings can be substituted.

As used herein (unless otherwise specified), the term “cycloalkyl” means a monovalent, monocyclic or bicyclic, 3, 4, 5, 6, 7, 8, 9, 10, 11, or 12 membered hydrocarbon group. The rings can be saturated or partially unsaturated. Examples of cycloalkyl groups include, but are not limited to, cyclopropyl, cyclobutyl, cyclopentyl, cyclohexyl, cycloheptyl, cyclooctyl, and bicycloalkyls (e.g., bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds). For a monocyclic cycloalkyl, the ring is not aromatic. For a bicyclic cycloalkyl, if one ring is aromatic, then the other is not aromatic. For a bicyclic cycloalkyl that is designated as substituted, one or both rings can be substituted.

As used herein (unless otherwise specified), the term “halogen” means monovalent Cl, F, Br, or I.

As used herein (unless otherwise specified), the term “hetero atom” means an atom selected from nitrogen atom, oxygen atom, or sulfur atom.

As used herein (unless otherwise specified), the terms “hydroxy” or “hydroxyl” indicates the presence of a monovalent —OH group.

As used herein (unless otherwise specified), the term “substituted” (e.g., as in substituted alkyl) means that one or more hydrogen atoms of a chemical group (with one or more hydrogen atoms) can be replaced by one or more non-hydrogen substituents selected from the specified options. The replacement can occur at one or more positions. The term “optionally substituted” means that one or more hydrogen atoms of a chemical group (with one or more hydrogen atoms) can be, but is not required to be, substituted.

In some embodiments, the compounds of the invention can be in the form of salts, optical and geometric isomers, and salts of isomers (e.g., as discussed herein). In other embodiments, the compounds can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). In yet other embodiments, simple derivatives of the compounds (e.g., ethers, esters, or amides) which can sometimes have desirable retention and release characteristics and which may also be easily hydrolyzed by body pH, enzymes, or other suitable means, can be employed.

Some compounds of the invention can have one or more chiral centers and can exist in and be isolated in optically active and racemic forms, for any of the one or more chiral centers. Some compounds can exhibit polymorphism. The compounds of the present invention (e.g., Formula I) encompass any optically active, racemate, stereoisomer form, polymorphism, or mixtures thereof. If a chiral center does not provide an indication of its configuration (i.e., R or S) in a chemical structure, it should be considered to represent R, S or a racemate.

Boron Compounds and Compositions including Pharmaceutical Compositions

Some embodiments of the invention include boron compounds selected from Formula (I) (i.e., Formula (I) is Formula (Ia) and Formula (Ib)), salts of Formula (I), optical isomers of Formula (I), geometric isomers of Formula (I), salts of optical isomers of Formula (I), salts of geometric isomers of Formula (I), and derivatives thereof,

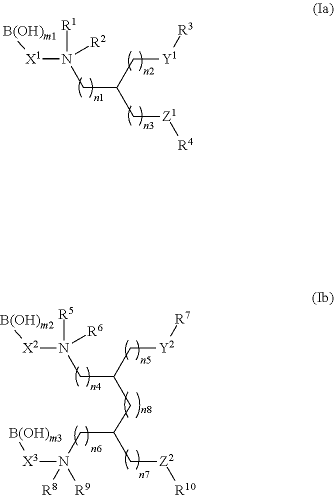

In certain embodiments, the nitrogen in Formula (Ia) (i.e., attached to X¹, R¹, and R²) can be positively charged. In other embodiments, the nitrogen in Formula (Ib) (i.e., attached to X², R⁵, and R⁶) can be positively charged. In some embodiments, the other nitrogen in Formula (Ib) (i.e., attached to X³, R⁸, and R⁹) can be positively charged. In still other embodiments, both nitrogens in Formula (Ib) (i.e., the nitrogen attached to X², R⁵, and R⁶, and the nitrogen attached to X³, R⁸, and R⁹) can be positively charged.

In certain embodiments, the boron in Formula (Ia) can be negatively charged. In other embodiments, one boron in Formula (Ib) can be negatively charged. In still other embodiments, both borons in Formula (Ib) can be negatively charged.

In some embodiments, the boron compound is selected from Formula (Ia1) and (Ia2), salts of Formula (Ia1) and (Ia2), optical isomers of Formula (Ia1) and (Ia2), geometric isomers of Formula (Ia1) and (Ia2), salts of optical isomers of Formula (Ia1) and (Ia2), salts of geometric isomers of Formula (Ia1) and (Ia2), and derivatives thereof

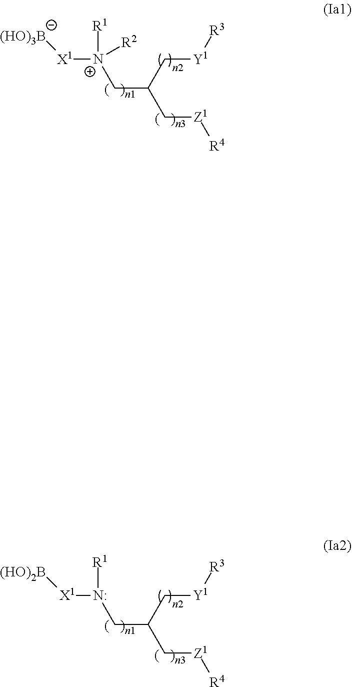

In other embodiments, X¹, X², and X³ can be the same or different and can be —C(R^{a})(R^{b})—, —C(R^{a})(R^{b})—C(R^{c})(R^{d})—, or —C(R^{a})(R^{b})—C(R^{c})(R^{d})—C(R^{e})(R^{f})—, where R^{a}, R^{b}, R^{c}, R^{d}, R^{e}, and R^{f} can be the same or different if they are on different X groups. In other embodiments, X¹, X², and X³ can be —C(R^{a})(R^{b})—.

In certain embodiments, R^{a}, R^{b}, R^{c}, R^{d}, R^{e}, and R^{f} can be the same or different and can be H, halogen (e.g., F, Cl, Br, or I), —CN, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, or —OCF₃. In certain embodiments, R^{a}, R^{b}, R^{c}, R^{d}, R^{e}, and R^{f} can be H.

In other embodiments, R¹, R², R⁵, R⁶, R⁸, and R⁹ can be the same or different and can be lone pair of electrons (i.e., a lone pair of electrons because it is not bonded to an atom), H, hydroxy (—OH), methanoyl (—COH), nitro (—NO₂), —NH₂, —N(CH₃)₂, cyano (—CN), sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, —OCF₃, C₁-C₁₀ alkyl, C₂-C₁₀ alkenyl, C₂-C₁₀ alkynyl, or C₁-C₉ alkoxy, which methanoyl (—COH), —NH₂, —N(CH₃)₂, —CONH₂, —CON(CH₃)₂, —C(O)(CH₃), —C(O)(C₂H₅), —C(O)(C₃H₇), C₁-C₁₀ alkyl, C₂-C₁₀ alkenyl, C₂-C₁₀ alkynyl, C₁-C₉ alkoxy can optionally be substituted with one or more of halogen, oxo (=O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluoronated alkyl, —CF₃, —OCF₃, or C₁-C₃ alkoxy. In certain embodiments, at least one of R¹ and R² is not a lone pair of electrons. In some embodiments, at least one of R⁵ and R⁶ is not a lone pair of electrons. In certain embodiments, at least one of R⁸ and R⁹ is not a lone pair of electrons. In other embodiments, R¹ and R² can be H, methyl, or ethyl. In still other embodiments, R⁵ and R⁶ can be H, methyl, or ethyl. In yet other embodiments, R⁸ and R⁹ can be H, methyl, or ethyl. In Formula (Ia2), R¹ is not a lone pair of electrons.

In certain embodiments, m1 can be 2 or 3.

In certain embodiments, m2 can be 2 or 3.

In certain embodiments, m3 can be 2 or 3.

In some embodiments, n1 and n4 can be the same or different and can be 1, 2, 3, 4, 5, 6, 7, 8, or 9. In certain embodiments, n1 and n4 can be the same or different and can be 1, 2, 3, or 4. In certain embodiments, n1 and n4 can be the same or different and can be 1.

In some embodiments, n6 can be 0, 1, 2, 3, 4, 5, 6, 7, 8, or 9. In certain embodiments, n6 can be 0, 1, 2, or 3.

In other embodiments, n2, n3, n5, and n7 can be the same or different and can be 0, 1, 2, 3, or 4. In some embodiments, n2, n3, n5, and n7 can be the same or different and can be 0, 1, or 2.

In certain embodiments, n8 can be 0, 1, 2, or 3. In other embodiments, n8 can be 0 or 1.

In other embodiments, Y¹ and Y² can be the same or different and can be —CH₂—, —O—(CO)—, —(CO)—O—, —O—(N═CH)—, —(CH═N)—O—, —O—, —N(R^{g})—(CO)—, or —(CO)—N(R^{g})—, where R^{g} can be the same or different if they are on different Y groups. In some embodiments, Y¹ and Y² can be the same or different and can be —O—(CO)—, —(CO)—O—, —O—(N═CH)—, —(CH═N)—O—, or —O—. In other embodiments, Y¹ and Y² can be the same or different and can be —O—(CO)—, —O—(N═CH)—, or —O—. In certain embodiments, Y¹ and Y² can be —O—(CO)—.

In some embodiments, Z¹ and Z² can be the same or different and can be —CH₂—, —O—(CO)—, —(CO)—O—, —O—(N═CH)—, —(CH═N)—O—, —O—, —N(R^{h})—(CO)—, or —(CO)—N(R^{h})—, where R^{h} can be the same or different if they are on different Z groups. In some embodiments, Z¹ and Z² can be the same or different and can be —O—(CO)—, —(CO)—O—, —O—(N═CH)—, —(CH═N)—O—, or —O—. In other embodiments, Z¹ and Z² can be the same or different and can be —O—(CO)—, —O—(N═CH)—, or —O—. In certain embodiments, Z¹ and Z² can be —O—(CO)—.

In other embodiments, R⁹ and R^{h} can be the same or different and can be H, methyl, ethyl, propyl, or butyl. In certain embodiments, R^{g} and R^{h} can be the same or different and can be H, methyl, or ethyl.

In certain embodiments, R³, R⁴, R⁷ and R¹⁰ can be the same or different and can be C₆-C₃₀ alkyl, C₆-C₃₀ alkenyl, or C₆-C₃₀ alkynyl. In some embodiments, R³, R⁴, R⁷ and R¹⁰ can be the same or different and can be C₁₂-C₁₈ alkyl, C₁₂-C₁₈ alkenyl, or C₆-C₃₀ alkynyl. In other embodiments, R³, R⁴, R⁷ and R¹⁰ can be the same or different and can be C₁₂-C₁₈ alkyl or C₁₂-C₁₈ alkenyl. In still other embodiments, R³ and R⁴ can be the same and can be C₁₃ alkyl. In yet other embodiments, R³ and R⁴ can be the same and can be C₁₇ alkenyl (A9). In still other embodiments, R⁷ and R¹⁰ can be the same and can be C₁₃ alkyl. In yet other embodiments, R⁷ and R¹⁰ can be the same and can be C₁₇ alkenyl (A9).

In some embodiments, X¹ is —C(R^{a})(R^{b})—, n1 is 1, n2 is 0, n3 is 1, Y¹ is —O—(CO)—, or Z¹ is —O—(CO)—, or a combination thereof. In some embodiments, X¹ is —CH₂—, R¹ is —CH₃, R² is —CH₃, n1 is 1, n2 is 0, n3 is 1, Y¹ is —O—(CO)—, or Z¹ is —O—(CO)—, or a combination thereof.

In some embodiments, X² is —C(R^{a})(R^{b})—, X³ is —C(R^{a})(R^{b})—, n4 is 1, n6 is 0, n5 is 0, n7 is 0, n8 is 0, Y² is —O—(CO)—, or Z² is —O—(CO)—, or a combination thereof. In some embodiments, X² is —CH₂—, X³ is —CH₂—, R⁵ is —CH₃, R⁶ is —CH₃, R⁸ is —CH₃, R⁹ is —CH₃, n4 is 1, n6 is 0, n5 is 0, n7 is 0, n8 is 0, Y² is —O—(CO)—, or Z² is —O—(CO)—, or a combination thereof.

In some embodiments, a pKₐ of the boron compound is 5-8 or 6-7.

In other embodiments, the boron compound is DMDBH, DODBH or DMTBH.

TABLE 1

| Formula # | |
|---|---|
| (other | |
| identifiers) | Compound Structure |
| I-1 (DMDBH) | 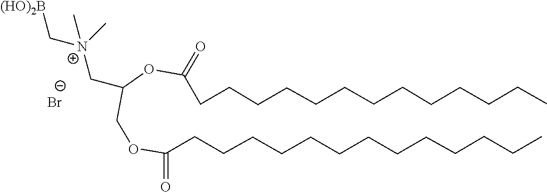 [SMILES: CCCCCCCCCCCCCC(=O)OCC(C[N+](C)(C)CB(O)O)OC(=O)CCCCCCCCCCCCC ; [Br-]] |
| I-2 (DODBH) | 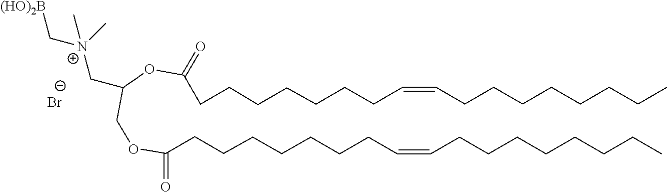 [SMILES: CCCCCCCC/C=C\CCCCCCCC(=O)OCC(C[N+](C)(C)CB(O)O)OC(=O)CCCCCCC/C=C\CCCCCCCC ; [Br-]] |
| I-3 (DMTBH) | 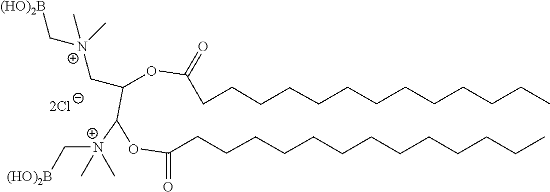 |
| I-4 | 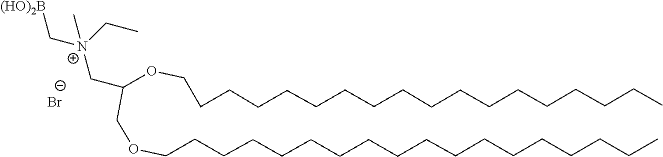 [SMILES: CCCCCCCCCCCCCCCCCCOCC(C[N+](C)(CC)CB(O)O)OCCCCCCCCCCCCCCCCCC ; [Br-]] |
| I-5 | 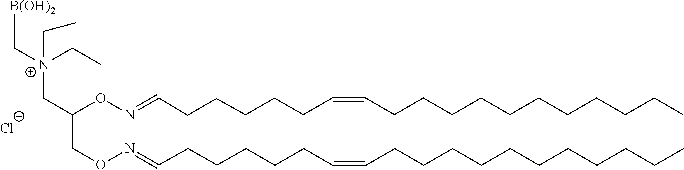 [SMILES: [Cl-] ; CCCCCCCCCCC/C=C\CCCCC/C=N/OCC(C[N+](CC)(CC)CB(O)O)O/N=C/CCCCC/C=C\CCCCCCCCCCC] |
| I-6 | 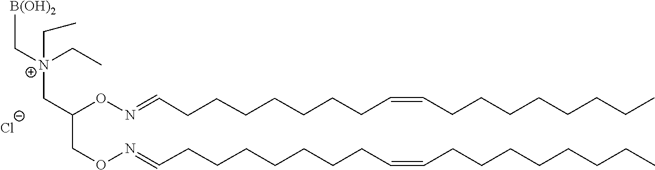 [SMILES: [Cl-] ; CCCCCCCC/C=C\CCCCCCC/C=N/OCC(C[N+](CC)(CC)CB(O)O)O/N=C/CCCCCCC/C=C\CCCCCCCC] |
| I-7 | 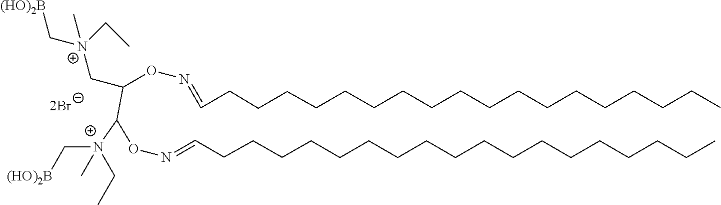 |
| I-8 | 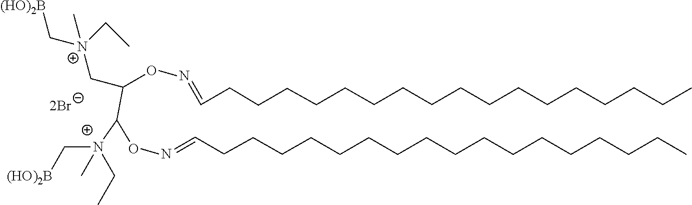 |
| I-9 | 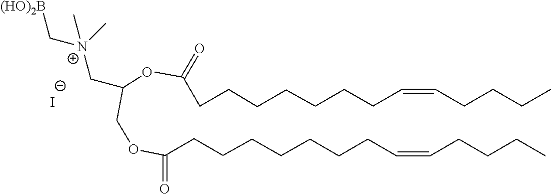 [SMILES: CCCC/C=C\CCCCCCCC(=O)OCC(C[N+](C)(C)CB(O)O)OC(=O)CCCCCCC/C=C\CCCC ; [I-]] |
| I-10 | 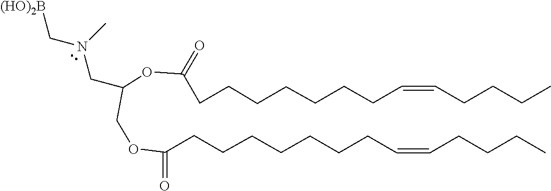 [SMILES: CCCC/C=C\CCCCCCCC(=O)OCC(CN(C)CB(O)O)OC(=O)CCCCCCC/C=C\CCCC] |
| I-11 | 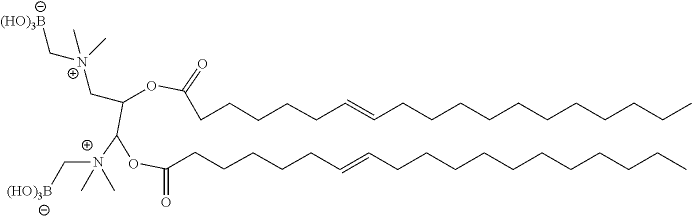 |
| I-12 | 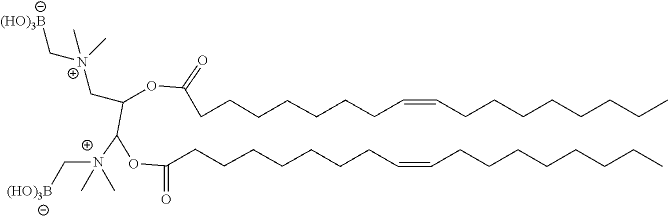 |
| I-13 | 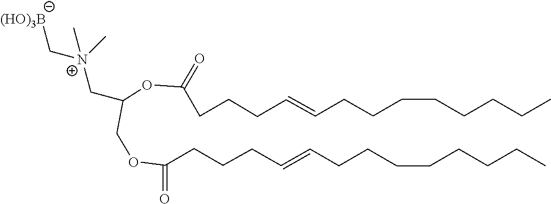 |
| I-14 | 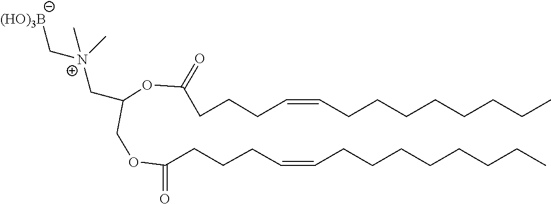 |
| I-15 | 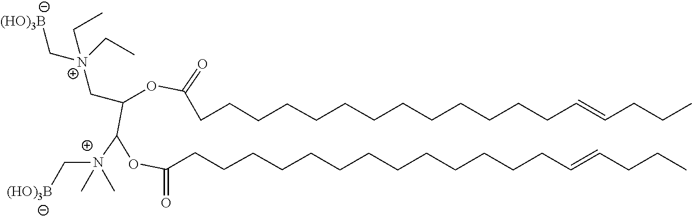 |
| I-16 | 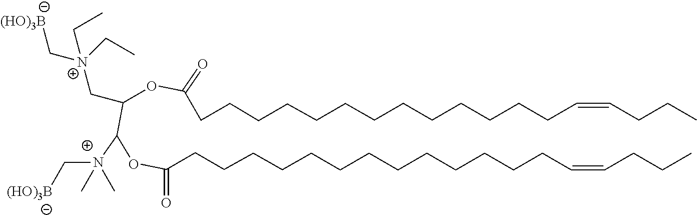 |
| I-17 | 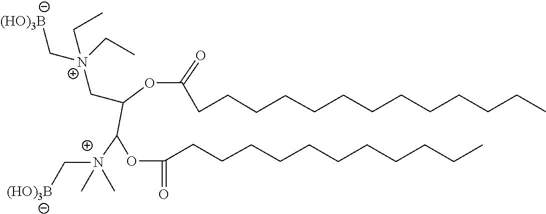 |

In some embodiments, one or more of compounds I-1, I-2, I-3, I-4, I-5, I-6, I-7, I-8, I-9, I-10, I-11, I-12, I-13, I-14, I-15, I-16, or I-17 are excluded from the compounds of the invention.

In some embodiments, the compounds of the invention include one or more of I-1, I-2, I-3, I-4, I-5, I-6, I-7, I-8, I-9, I-10, I-11, I-12, I-13, I-14, I-15, I-16, or I-17. In some embodiments, the compounds of the invention include I-1, I-2, I-3, I-4, I-5, I-6, I-7, I-8, I-9, I-10, I-11, I-12, I-13, I-14, I-15, I-16, and I-17.

One or more boron compounds can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

One or more boron compounds can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%. Some embodiments of the present invention include compositions comprising one or more boron compounds. In certain embodiments, the composition is a pharmaceutical composition (e.g., in a lipid particle composition or a lipoplex composition), such as compositions that are suitable for administration to animals (e.g., mammals, primates, monkeys, humans, canine, porcine, mice, rabbits, or rats).

Lipid Particles, Lipoplexes, and Their Compositions including Pharmaceutical Compositions

Some embodiments of the invention include lipid particles comprising a boron compound (e.g., Formulas, (Ia), (Ib), (Ia1), or (Ia2)). Examples of lipid particles include lipid nanoparticles, liposomes, and liposome formulations. The lipid particles can comprise any suitable molecules, such as but not limited to a molecule (e.g., lipid) with a diol, a molecule (e.g., lipid) with a 1,2 diol (e.g., OEL4, iOEL4, eiOEL4, or OEL5), a molecule (e.g., lipid) with a 1,3 diol, a sugar, an amino acid, a peptide, a polypeptide, a polynucleotide, a PEG polymer, or any suitable lipid. Suitable lipids can include but are not limited to OEL4, iOEL4, eiOEL4, OEL5, fatty acids (characterized by a carboxyl group and an acyl chain), Glycerolipids (characterized by the presence of a glycerol backbone with one—monoacylglycerols (MAG), two—diacylglycerols (DAG) or three—triacylglycerols (TAG) ester linked fatty acyl chains), glycerophospholipids (GPL) (characterized by a glyceryl backbone with two ester-linked acyl chains and a phosphate-linked polar headgroup—GPLs include phosphatidylcholines (PC), phosphatidylethanolamines (PE), phosphatidylserines (PS), phosphatidylglycerols (PG), phosphatidylinositols (PI), inositol, and phosphatidic acids), Lysoglycerophospholipids (LGPL) (LGPLs are missing one of the acyl chains at the glycerol backbone, e.g., at the C2 position—LGPLs include lysophospahtidylcholines (LysoPC), lysophosphatidylethanolamines (LysoPE), lysophosphatidylserines (LysoPS), lysophosphatidylglycerols (LysoPG), lysophosphatidylinositols (LysoPI), lysoinositol, and lysophosphatidic acids), sphingolipids (SPL) (characterized by a sphingosine base backbone with a trans double bond between C4 and C5 of an acyl chain linked to the amino group via an amide linkage), Bis(monoacylglycero)phosphate (BMP), Ceramides, Gangliosides, Sterols, cholesterol, Prenols, Saccharolipids, Polyketides, and PEGylation (e.g., with PEG 100, PEG 200, PEG 250, PEG 350, PEG 500, PEG 1000, PEG 2000, or PEG 3000) of any of the aforementioned. Examples of suitable molecules for lipid particles include but are not limited to OEL4, iOEL4, eiOEL4, OEL5, DOPE, DSPE-PEG350, PBA, and cholesterol. Other examples of suitable molecules for lipid particles include but are not limited to those disclosed in Gupta et al. (2015) “Oxime ether lipids containing hydroxylated head groups are more superior siRNA delivery agents than their nonhydroxylated counterparts” Nanomedicine (Lond.), Vol. 10, No. 18, pp. 2805-2818, which is herein incorporated by reference in its entirety; Li et al. (2022) “Payload distribution and capacity of mRNA lipid nanoparticles” Nature Communications, Vol. 13, Article 5561 (13 pages), which is herein incorporated by reference in its entirety; Puri et al. (2022) “Stealth oxime ether lipid vesicles promote delivery of functional DsiRNA in human lung cancer A549 tumor bearing mouse xenografts” Nanomedicine: Nanotechnology, Biology, and Medicine, Vol. 44, Article 102572 (15 pages), which is herein incorporated by reference in its entirety; Roces et al. (2020) “Manufacturing Considerations for the Development of Lipid Nanoparticles Using Microfluidics” Pharmaceutics, Vol. 12, Article 1095 (19 pages), which is herein incorporated by reference in its entirety; Schoenmaker et al. (2021) “mRNA-lipid nanoparticle COVID-19 vaccines: Structure and stability” International Journal of Pharmaceutics, Vol. 601, Article 120586 (13 pages), which is herein incorporated by reference in its entirety.

In some embodiments, the amount of the boron compound in the lipid particle is 0.01 to 50 mol % or 0.1 to 25 mol % or 1 to 10 mol %.

In certain embodiments, the boron compound can react with a lipid particle component in the lipid particle to form one or more covalent bonds between the boron compound and the lipid particle component. In other embodiments, lipid particle component forming the one or more covalent bonds can be any suitable molecule, including but not limited to a molecule with a diol, a molecule with a 1,2 diol (e.g., OEL4, iOEL4, eiOEL4, or OEL5), a molecule with a 1,3 diol, a sugar, an amino acid, a peptide, a polypeptide, a polynucleotide, and a PEG polymer. In certain embodiments, the lipid particle component is not a boron compound. In certain embodiments, the lipid particle component is a molecule with a diol (e.g., a molecule (e.g., lipid) with a 1,2 diol or a molecule (e.g., lipid) with a 1,3 diol). In other embodiments, the lipid particle component is OEL4, iOEL4, eiOEL4, or OEL5.

In other embodiments, the diameter of the lipid particle is 30-250 nm, 50-110 nm, 60-105 nm, or 65-100 nm. In some embodiments, the diameter of the lipid particle is decreased by at least 10%, by at least 20%, by at least 30%, by at least 40%, by at least 50%, by at least 60%, by at least 70%, or by at least 80%, compared to the lipid particle without addition of the boron compound.

The lipid particles can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising the lipid particles. In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

Certain embodiments of the present invention include methods to prepare lipid particles. Any suitable method can used to prepare lipid particles, including but not limited to those disclosed herein and those disclosed in Gupta et al. (2015) “Oxime ether lipids containing hydroxylated head groups are more superior siRNA delivery agents than their nonhydroxylated counterparts” Nanomedicine (Lond.), Vol. 10, No. 18, pp. 2805-2818, which is herein incorporated by reference in its entirety; Li et al. (2022) “Payload distribution and capacity of mRNA lipid nanoparticles” Nature Communications, Vol. 13, Article 5561 (13 pages), which is herein incorporated by reference in its entirety; Puri et al. (2022) “Stealth oxime ether lipid vesicles promote delivery of functional DsiRNA in human lung cancer A549 tumor bearing mouse xenografts” Nanomedicine: Nanotechnology, Biology, and Medicine, Vol. 44, Article 102572 (15 pages), which is herein incorporated by reference in its entirety; Roces et al. (2020) “Manufacturing Considerations for the Development of Lipid Nanoparticles Using Microfluidics” Pharmaceutics, Vol. 12, Article 1095 (19 pages), which is herein incorporated by reference in its entirety; Schoenmaker et al. (2021) “mRNA-lipid nanoparticle COVID-19 vaccines: Structure and stability” International Journal of Pharmaceutics, Vol. 601, Article 120586 (13 pages), which is herein incorporated by reference in its entirety.

Some embodiments of the invention include lipoplexes comprising (a) a boron compound (e.g., Formulas, (Ia), (Ib), (Ia1), or (Ia2)) or a lipid particle (e.g., as disclosed herein) and (b) a nucleic acid molecule (e.g., ssDNA, dsDNA, ssRNA dsRNA, or mRNA), such as a nucleic acid molecule having a molecular weight (in daltons) of no more than 10,000,000, no more than 2,000,000, no more than 1,000,000, no more than 500,000, no more than 100,000, or no more than 10,000. The molecular weight of the nucleic acid molecule can be determined using gel permeation chromatography (GPC). The nucleic acid molecule can be used to inhibit gene or protein expression, enhance gene or protein expression, or provide expression of a protein (e.g., not otherwise found in the targeted cell, organ, or organism).

The lipoplexes can comprise any suitable nucleic acid molecule, including but not limited to those disclosed herein, or those disclosed in Gupta et al. (2015) “Oxime ether lipids containing hydroxylated head groups are more superior siRNA delivery agents than their nonhydroxylated counterparts” Nanomedicine (Lond.), Vol. 10, No. 18, pp. 2805-2818, which is herein incorporated by reference in its entirety; Li et al. (2022) “Payload distribution and capacity of mRNA lipid nanoparticles” Nature Communications, Vol. 13, Article 5561 (13 pages), which is herein incorporated by reference in its entirety; Puri et al. (2022) “Stealth oxime ether lipid vesicles promote delivery of functional DsiRNA in human lung cancer A549 tumor bearing mouse xenografts” Nanomedicine: Nanotechnology, Biology, and Medicine, Vol. 44, Article 102572 (15 pages), which is herein incorporated by reference in its entirety; Roces et al. (2020) “Manufacturing Considerations for the Development of Lipid Nanoparticles Using Microfluidics” Pharmaceutics, Vol. 12, Article 1095 (19 pages), which is herein incorporated by reference in its entirety; Schoenmaker et al. (2021) “mRNA-lipid nanoparticle COVID-19 vaccines: Structure and stability” International Journal of Pharmaceutics, Vol. 601, Article 120586 (13 pages), which is herein incorporated by reference in its entirety.

The lipoplexes can comprise any suitable molecules (such as those disclosed herein for lipid particles), such as but not limited to a molecule (e.g., lipid) with a diol, a molecule (e.g., lipid) with a 1,2 diol (e.g., OEL4, iOEL4, eiOEL4, or OEL5), a molecule (e.g., lipid) with a 1,3 diol, a sugar, an amino acid, a peptide, a polypeptide, a polynucleotide, a PEG polymer, or any suitable lipid. Suitable lipids can include but are not limited to OEL4, iOEL4, eiOEL4, OEL5, fatty acids (characterized by a carboxyl group and an acyl chain), Glycerolipids (characterized by the presence of a glycerol backbone with one—monoacylglycerols (MAG), two—diacylglycerols (DAG) or three—triacylglycerols (TAG) ester linked fatty acyl chains), glycerophospholipids (GPL) (characterized by a glyceryl backbone with two ester-linked acyl chains and a phosphate-linked polar headgroup—GPLs include phosphatidylcholines (PC), phosphatidylethanolamines (PE), phosphatidylserines (PS), phosphatidylglycerols (PG), phosphatidylinositols (PI), inositol, and phosphatidic acids), Lysoglycerophospholipids (LGPL) (LGPLs are missing one of the acyl chains at the glycerol backbone, e.g., at the C2 position—LGPLs include include lysophospahtidylcholines (LysoPC), lysophosphatidylethanolamines (LysoPE), lysophosphatidylserines (LysoPS), lysophosphatidylglycerols (LysoPG), lysophosphatidylinositols (LysoPI), lysoinositol, and lysophosphatidic acids), sphingolipids (SPL) (characterized by a sphingosine base backbone with a trans double bond between C4 and C5 of an acyl chain linked to the amino group via an amide linkage), Bis(monoacylglycero)phosphate (BMP), Ceramides, Gangliosides, Sterols, cholesterol, Prenols, Saccharolipids, Polyketides, and PEGylation (e.g., with PEG 100, PEG 200, PEG 250, PEG 350, PEG 500, PEG 1000, PEG 2000, or PEG 3000) of any of the aforementioned. Examples of suitable molecules for lipid particles include but are not limited to OEL4, iOEL4, eiOEL4, OEL5, DOPE, DSPE-PEG350, PBA, and cholesterol. Other examples of suitable molecules for lipid particles include but are not limited to those disclosed in Gupta et al. (2015) “Oxime ether lipids containing hydroxylated head groups are more superior siRNA delivery agents than their nonhydroxylated counterparts” Nanomedicine (Lond.), Vol. 10, No. 18, pp. 2805-2818, which is herein incorporated by reference in its entirety; Li et al. (2022) “Payload distribution and capacity of mRNA lipid nanoparticles” Nature Communications, Vol. 13, Article 5561 (13 pages), which is herein incorporated by reference in its entirety; Puri et al. (2022) “Stealth oxime ether lipid vesicles promote delivery of functional DsiRNA in human lung cancer A549 tumor bearing mouse xenografts” Nanomedicine: Nanotechnology, Biology, and Medicine, Vol. 44, Article 102572 (15 pages), which is herein incorporated by reference in its entirety; Roces et al. (2020) “Manufacturing Considerations for the Development of Lipid Nanoparticles Using Microfluidics” Pharmaceutics, Vol. 12, Article 1095 (19 pages), which is herein incorporated by reference in its entirety; Schoenmaker et al. (2021) “mRNA-lipid nanoparticle COVID-19 vaccines: Structure and stability” International Journal of Pharmaceutics, Vol. 601, Article 120586 (13 pages), which is herein incorporated by reference in its entirety.

In some embodiments, the amount of the boron compound in the lipoplex is 0.01 to 50 mol % or 0.1 to 25 mol % or 1 to 10 mol %.

In certain embodiments, the boron compound reacts with a lipoplex component in the lipoplex to form one or more covalent bonds between the boron compound and the lipid particle component. In other embodiments, lipoplex component can be any suitable molecule, including but not limited to a molecule with a diol, a molecule with a 1,2 diol (e.g., OEL4, iOEL4, eiOEL4, or OEL5), a molecule with a 1,3 diol, a sugar, an amino acid, a peptide, a polypeptide, a polynucleotide, and a PEG polymer. In certain embodiments, the lipoplex component is not a boron compound. In certain embodiments, the lipoplex component is a molecule with a diol (e.g., a molecule (e.g., lipid) with a 1,2 diol or a molecule (e.g., lipid) with a 1,3 diol). In other embodiments, the lipoplex component is OEL4, iOEL4, eiOEL4, or OEL5.

In other embodiments, the diameter of the lipoplex is 30-250 nm, 50-110 nm, 60-105 nm, or 65-100 nm. In some embodiments, the diameter of the lipoplex is decreased by at least 10%, by at least 20%, by at least 30%, by at least 40%, by at least 50%, by at least 60%, by at least 70%, or by at least 80%, compared to the lipoplex without addition of the boron compound.

The lipoplexes can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising the lipoplexes. In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

Certain embodiments of the present invention include methods to prepare lipoplexes. Any suitable method can used to prepare lipoplexes, including but not limited to those disclosed herein and those disclosed in Gupta et al. (2015) “Oxime ether lipids containing hydroxylated head groups are more superior siRNA delivery agents than their nonhydroxylated counterparts” Nanomedicine (Lond.), Vol. 10, No. 18, pp. 2805-2818, which is herein incorporated by reference in its entirety; Li et al. (2022) “Payload distribution and capacity of mRNA lipid nanoparticles” Nature Communications, Vol. 13, Article 5561 (13 pages), which is herein incorporated by reference in its entirety; Puri et al. (2022) “Stealth oxime ether lipid vesicles promote delivery of functional DsiRNA in human lung cancer A549 tumor bearing mouse xenografts” Nanomedicine: Nanotechnology, Biology, and Medicine, Vol. 44, Article 102572 (15 pages), which is herein incorporated by reference in its entirety; Roces et al. (2020) “Manufacturing Considerations for the Development of Lipid Nanoparticles Using Microfluidics” Pharmaceutics, Vol. 12, Article 1095 (19 pages), which is herein incorporated by reference in its entirety; Schoenmaker et al. (2021) “mRNA-lipid nanoparticle COVID-19 vaccines: Structure and stability” International Journal of Pharmaceutics, Vol. 601, Article 120586 (13 pages), which is herein incorporated by reference in its entirety. [00109]“Therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any method known in the art, such as measurement of antibody titers.

In some embodiments, the lipid particles or lipoplexes can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, intrathecal administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof.

In certain embodiments, pharmaceutical compositions can be formulated to release the active ingredient (e.g., lipid particles or lipoplexes or the nucleic acid in the lipoplex) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Other embodiments of the invention can include methods of administering or treating an organism, which can involve treatment with an amount of the lipoplexes (or the nucleic acid in the lipoplex) that is effective to treat the disease, condition, or disorder that the organism has, or is suspected of having, or is susceptible to, or to bring about a desired physiological effect. In some embodiments, the composition or pharmaceutical composition (e.g., a vaccine) comprises lipoplexes which can be administered to an animal (e.g., mammals, primates, monkeys, or humans) in an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the compounds of the invention can be administered in combination with one or more other therapeutic agents for a given disease, condition, or disorder.

In some embodiments, the compositions can include a unit dose of the lipoplexes (or the nucleic acid in the lipoplex) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

### Methods of Use, Administration Routes, Treatments of Disease, and Vaccinations

Some embodiments of the invention include methods for providing nucleic acid to a cell (e.g., plant, cell, animal cell, mammalian cell, or human cell) comprising one or more administrations to the cell of one or more compositions comprising the lipoplex (e.g., as discussed herein). In certain embodiments, the compositions may be the same or different if there is more than one administration. In some embodiments, the method silences a gene in a cell or the added nucleic acid results in the expression of a protein or polypeptide (e.g., a portion of a protein). In other embodiments, the cell is in vivo, ex vivo, or in vitro.

Some embodiments of the invention include methods for providing an animal with a lipoplex (e.g., as discussed herein), comprising one or more administrations to the animal of one or more compositions (e.g., pharmaceutical compositions) comprising the lipoplex (e.g., as disclosed herein). In certain embodiments, the compositions may be the same or different if there is more than one administration. In other embodiments, at least one of the one or more compositions further comprises a formulary ingredient.

Some embodiments of the invention include treating an animal for a disease (e.g., cancers or infections) comprising one or more administrations to the animal of one or more compositions comprising the lipoplex (e.g., as disclosed herein). In certain embodiments, the compositions may be the same or different if there is more than one administration. In other embodiments, at least one of the one or more compositions further comprises a formulary ingredient.

Some embodiments of the invention include inducing an immune response in an animal for a disease (e.g., cancers or infections) or vaccinating an animal for a disease (e.g., cancers or infections), comprising one or more administrations to the animal of one or more compositions comprising the lipoplex (e.g., as disclosed herein). In certain embodiments, the compositions may be the same or different if there is more than one administration. In other embodiments, at least one of the one or more compositions further comprises a formulary ingredient.

The lipoplexes of the invention (e.g., those disclosed herein) can be administered to animals by any number of suitable administration routes or formulations. The lipoplexes of the invention can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of the lipoplexes of the invention can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, intrathecal administration, subcutaneous administration, topical administration, intradermal administration, oral administration, sublingual administration, intranasal administration, or intramuscular administration. The choice of administration route can depend on the lipoplex identity (e.g., the composition (e.g., the boron compound and/or the nucleic acid) of the lipoplex or the physical and chemical properties of the lipoplexes) as well as the age and weight of the animal, the particular disease, and the severity of the disease. Of course, combinations of administration routes can be administered, as desired.

Some embodiments of the invention include a method for providing a subject with a composition comprising a lipoplex described herein (e.g., a pharmaceutical composition) which comprises one or more administrations of one or more such compositions; the compositions may be the same or different if there is more than one administration.

Diseases that can be treated in an animal (e.g., mammals, porcine, canine, avian (e.g., chicken), bovine, feline, primates, monkeys, rabbits, and humans) using the lipoplexes include, but are not limited to infections (e.g., bacterial, viral or fungal infections, such as coronavirus) and cancers including but not limited to acute lymphoblastic leukemia, astrocytoma, basal cell carcinoma, bladder cancer, bone marrow cancer, breast cancer, chronic lymphocytic leukemia (CLL), CNS cancer, colon cancer, colorectal cancer, endometrial cancer, gastric cancer, glioblastoma, glioblastoma multiforme, glioma, gliosarcoma, hepatocellular carcinoma, kidney cancer, leukemia, liver cancer, lung cancer, lymphoma, melanoma, malignant nerve sheath tumors, medulloblastoma, meningioma, multiple myeloma, nasopharyngeal carcinoma, neuroblastoma, non-Hodgkin lymphoma, non-small cell lung cancer, oral cancer, ovarian cancer, pancreatic cancer, prostate cancer, rectal cancer, renal cancer, renal cell carcinoma, rhabdomyosarcoma, squamous cell carcinoma, stomach cancer, thyroid cancer, uterine cancer, cancers that can result in metastasis, cancers resulting from metastasis, or cancerous tumors thereof. Animals that can be treated include but are not limited to mammals, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., a prophylactic treatment).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating diseases (e.g., cancers or infections), where treating can include but is not limited to prophylactic treatment and therapeutic treatment. As such, treatment can include, but is not limited to: conferring protection against disease (e.g., cancers or infections); preventing disease (e.g., cancers or infections); reducing the risk of disease (e.g., cancers or infections); ameliorating or relieving symptoms of disease (e.g., cancers or infections); eliciting an immune response against a disease related element (e.g., a virus, bacteria, or fungus, or cancer cell) or an antigenic component thereof; inhibiting the development or progression of disease (e.g., cancers or infections); inhibiting or preventing the onset of symptoms associated with disease (e.g., cancers or infections); reducing the severity of disease (e.g., cancers or infections); and causing a regression of disease (e.g., cancers or infections) or one or more of the symptoms associated with disease (e.g., cancers or infections). In some embodiments, treating does not include prophylactic treatment (e.g., vaccination or otherwise preventing or ameliorating future disease).

Symptoms associated with cancer are known to those of ordinary skill in the art and can include those described herein and well-known to those of ordinary skill in the art. The presence of cancer can be assessed using methods known to those or ordinary skill in the art. In some cases, the presence of cancer can be determined using methods known to those of ordinary skill in the art.

Treatment of an animal can occur using any suitable administration method (such as those disclosed herein) and using any suitable amount of lipoplexes (such as those disclosed herein)(or the nucleic acid in the lipoplex). In some embodiments, methods of treatment comprise treating an animal for a disease (e.g., cancers or infections). Some embodiments of the invention include a method for treating a subject (e.g., an animal such as a human or primate) with a composition comprising a lipoplex described herein (e.g., a pharmaceutical composition) which comprises one or more administrations of one or more such compositions; the compositions may be the same or different if there is more than one administration.

In some embodiments, the method of treatment includes administering an effective amount of a composition comprising lipoplexes. As used herein, the term “effective amount” refers to a dosage or a series of dosages sufficient to affect treatment (e.g., to treat cancer) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of the lipoplex (or the nucleic acid in the lipoplex) (which can be administered to an animal such as mammals, primates, monkeys or humans) can be an amount of about 0.005 to about 50 mg/kg body weight, about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg.

In some embodiments, the treatments disclosed herein can include use of other drugs (e.g., antibiotics) or therapies for treating disease. For example, antibiotics can be used to treat infections and can be combined with lipoplxes to treat disease (e.g., infections). In other embodiments, intravenous immunoglobulin (IVIG) therapy can be used as part of the treatment regime (i.e., in addition to administration of lipoplexes) of cancer.

As used herein, “immunizing” and “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells))), and increased processing and presentation of antigen by antigen presenting cells. An immune response can be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade and/or activation of complement), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids)). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

In some embodiments, the treatment comprises vaccination (e.g., against cancer or infection by bacteria, virus, or fungus, such as coronavirus). In some aspects, vaccination comprises vaccinating an animal (e.g., mammals, primates, monkeys (e.g., macaque, rhesus macaque, pig tail macaque), humans, canine, feline, bovine, porcine, avian (e.g., chicken), mice, rabbits, and rats) against a disease (e.g., cancer or infection by bacteria, virus, or fungus, such as coronavirus). Any suitable administration methods or protocols can be used for vaccinating an animal. Some embodiments for vaccination include a method for providing a subject with a composition comprising a lipoplex described herein (or the nucleic acid in the lipoplex) (e.g., a pharmaceutical composition) which comprises one or more administrations of one or more such compositions; the compositions may be the same or different if there is more than one administration. For example, a single dose can be administered to a subject, or alternatively, two or more inoculations can take place with intervals of several weeks to several months. The extent and nature of the immune responses induced in the subject can be assessed using a variety of techniques generally known in the art. For example, sera can be collected from the subject and tested, for example, for infection-related DNA or RNA in a sera sample, detecting the presence of antibodies or antigenic fragments thereof using, for example, or monitoring a symptom associated with the infection. Relevant techniques are well described in the art, e.g., Coligan et al. Current Protocols in Immunology, John Wiley & Sons Inc. (1994), which is herein incorporated by reference in its entirety.

The timing of administration of the vaccine and the number of doses required for immunization can be determined from standard vaccine administration protocols. In some instances, a vaccine composition will be administered in two doses. The first dose will be administered at the elected date and a second dose will follow at one month from the first dose. A third dose can be administered if necessary, and desired time intervals for delivery of multiple doses of a particular lipoplex can be determined. In other embodiments, the lipoplex may be given as a single dose.

In some instances, for each recipient, the total vaccine amount necessary can be deduced from protocols for immunization with other vaccines. In some embodiments, the exact amount of lipoplex required can vary from subject to subject, depending on the species, age, weight and general condition of the subject, the particular fusion protein used, its mode of administration, and the like. In other embodiments, dosage will approximate that which is typical for the administration of other vaccines, and may be in the range of from about 1 ng/kg to about 1 mg/kg body weight, from about 10 ng/kg to about 15 mg/kg, or from about 10 ng/kg to about 100 mg/kg.

### Methods for Preparing Boron Compounds of Formula (Ia) and Salts Thereof

Some embodiments of the present invention include methods for the preparation of certain boron compounds of Formula (Ia) and salts thereof. In certain embodiments, a boron compound of Formula (Ia) (or their salts) can be prepared comprising the step of reacting a compound of Formula (IIa) to result in a compound of Formula (IIIa). Then, Formula (IIIa) is reacted to result in Formula (IVa), which is then reacted to result in Formula (Ia) or a salt thereof (e.g., using one or more synthetic steps). Unless otherwise specified, the moieties are defined as provided herein.

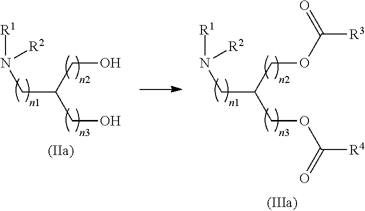

In some embodiments, Formula (IIa) can be reacted to provide Formula (IIIa) under the following conditions: Formula (IIa) can be mixed with R³—(O)C-(halogen) and R⁴—(O)C-(halogen) (e.g., myristoyl chloride, oleoyl chloride, or a combination thereof) in any suitable solvent (e.g., TEA, DMAP, and/or CH₂Cl₂) at any suitable temperature (e.g., 0° C. or rt) for any suitable period of time (e.g., 12 h). Formula (IIIa) can then optionally be recovered.

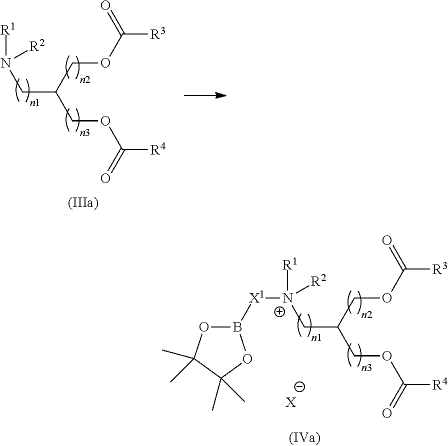

In some embodiments, Formula (IIIa) can be reacted to provide Formula (IVa) under the following conditions: Formula (IIIa) can be mixed with 2-(halomethyl)-4,4,5,5-tetramethyl-1,3,2-dioxaborolane (e.g., 2-(bromomethyl)-4,4,5,5-tetramethyl-1,3,2-dioxaborolane) in any suitable solvent (e.g., TEA, DMAP, and/or CH₂Cl₂) at any suitable temperature (e.g., 50° C.) for any suitable period of time (e.g., 8 h). Formula (IVa) can then optionally be recovered.

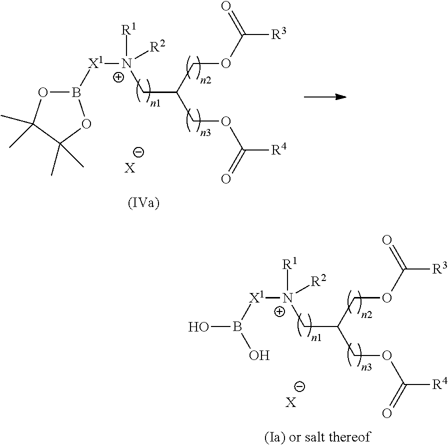

In some embodiments, Formula (IVa) can be reacted to provide Formula (Ta) or a salt thereof under the following conditions: Formula (IVa) can be mixed with NaOI₄ (or any suitable molecule to oxidatively create the B—OH groups) and HCl (2.0 M) in any suitable solvent (e.g., H₂O/THF) at any suitable temperature (e.g., 0° C.) for any suitable period of time (e.g., 12 h). Formula (Ia) or the salt thereof can then optionally be recovered.

Recovery can occur using any suitable method including but not limited to HPLC (e.g., reverse phase), LC, precipitation, centrifugation, column chromatography (e.g., size exclusion chromatography or ion exchange chromatography), use of silica gel, or combinations thereof.

In some embodiments, a method for the preparation of a boron compound of Formula (Ia) or salt thereof can comprise one or more of the above-mentioned steps. In certain embodiments, a method for preparing a compound of Formula (Ia) or salt thereof comprises
(a) reacting a compound of Formula (IIa) with R³—(O)C-(halogen) and R⁴—(O)C-(halogen) to result in a mixture comprising a compound of Formula (IIIa);
(b) reacting a compound of Formula (IIIa) with 2-(halomethyl)-4,4,5,5-tetramethyl-1,3,2-dioxaborolane (e.g., 2-(bromomethyl)-4,4,5,5-tetramethyl-1,3,2-dioxaborolane) to result in a mixture comprising a compound of Formula (IVa);
(c) reacting a compound of Formula (IVa) with any suitable molecule to oxidatively create the B—OH groups; and;
(d) recovering Formula (Ia) or salt thereof.

### Methods for Preparing Boron Compounds of Formula (Ib) or Salts Thereof

Some embodiments of the present invention include methods for the preparation of certain boron compounds of Formula (Ib) and salts thereof. In certain embodiments, a boron compound of Formula (Ib) (or their salts) can be prepared comprising the step of reacting a compound of Formula (IIb) to result in a compound of Formula (IIIb). Then, Formula (IIIb) is reacted to result in Formula (IVb), which is then reacted to result in Formula (Ib) or a salt thereof (e.g., using one or more synthetic steps). Unless otherwise specified, the moieties are defined as provided herein.

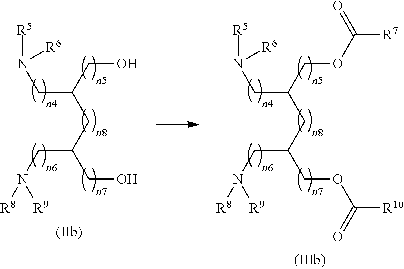

In some embodiments, Formula (IIb) can be reacted to provide Formula (IIIb) under the following conditions: Formula (IIb) can be mixed with R⁷—(O)C-(halogen) and R¹⁰—(O)C-(halogen) (e.g., myristoyl chloride, oleoyl chloride, or a combination thereof) in any suitable solvent (e.g., TEA, DMAP, and/or CH₂Cl₂) at any suitable temperature (e.g., 0° C. or rt) for any suitable period of time (e.g., 18 h). Formula (IIIb) can then optionally be recovered.

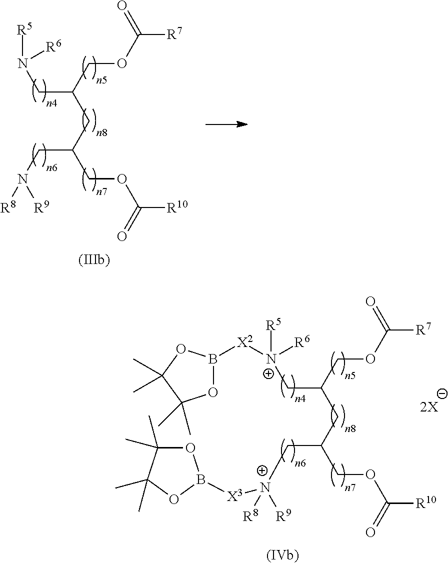

In some embodiments, Formula (IIIb) can be reacted to provide Formula (IVb) under the following conditions: Formula (IIIb) can be mixed with 2-(halomethyl)-4,4,5,5-tetramethyl-1,3,2-dioxaborolane pinacol ester (e.g., 2-(bromomethyl)-4,4,5,5-tetramethyl-1,3,2-dioxaborolane pinacol ester) in any suitable solvent (e.g., TEA, DMAP, and/or CH₂Cl₂) at any suitable temperature (e.g., 50° C.) for any suitable period of time (e.g., 24 h). Formula (IVb) can then optionally be recovered.

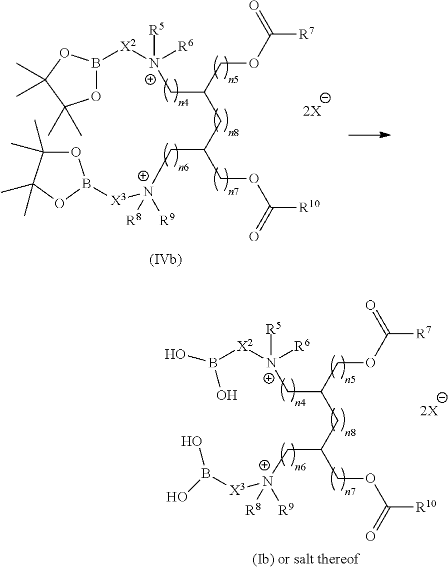

In some embodiments, Formula (IVb) can be reacted to provide Formula (Ib) or salt thereof under the following conditions: Formula (IVb) can be mixed with methyl boronic acid and HCl (2.0 M) in any suitable solvent (e.g., methanol) at any suitable temperature (e.g., rt) for any suitable period of time (e.g., 24 h). Formula (Ib) or salt thereof can then optionally be recovered.

Recovery can occur using any suitable method including but not limited to HPLC (e.g., reverse phase), LC, precipitation, centrifugation, column chromatography (e.g., size exclusion chromatography or ion exchange chromatography), use of silica gel, or combinations thereof.

In some embodiments, a method for the preparation of a boron compound of Formula (Ib) or salt thereof can comprise one or more of the above-mentioned steps. In certain embodiments, a method for preparing a boron compound of Formula (Ib) or salt thereof comprises
(a) reacting a compound of Formula (IIb) with R⁷—(O)C-(halogen) and R¹⁰—(O)C-(halogen) to result in a mixture comprising a compound of Formula (IIIb);
(b) reacting a compound of Formula (IIIb) with 2-(halomethyl)-4,4,5,5-tetramethyl-1,3,2-dioxaborolane pinacol ester (e.g., 2-(bromomethyl)-4,4,5,5-tetramethyl-1,3,2-dioxaborolane pinacol ester) to result in a mixture comprising a compound of Formula (IVb);
(c) reacting a compound of Formula (IVb) with any suitable molecule to oxidatively create the B—OH groups; and;
(d) recovering Formula (Ib) or salt thereof.

The presently-disclosed subject matter is further illustrated by the following specific but non-limiting examples. The following examples may include compilations of data that are representative of data gathered at various times during the course of development and experimentation related to the present invention.

### EXAMPLES

### Example Set A1—Boronic Compounds—Synthesis and Characterization

Additional experimental details for synthesis and characterization of DMDBH and DODBH can be found in Dr. Ibrahim's Ph.D. dissertation (Chapters 5 and 6) (Ibrahim, Faisal, “Micelles and lipid nanoparticles: catalysis and biomedical application.” (2021). Electronic Theses and Dissertations. Paper 3731.)

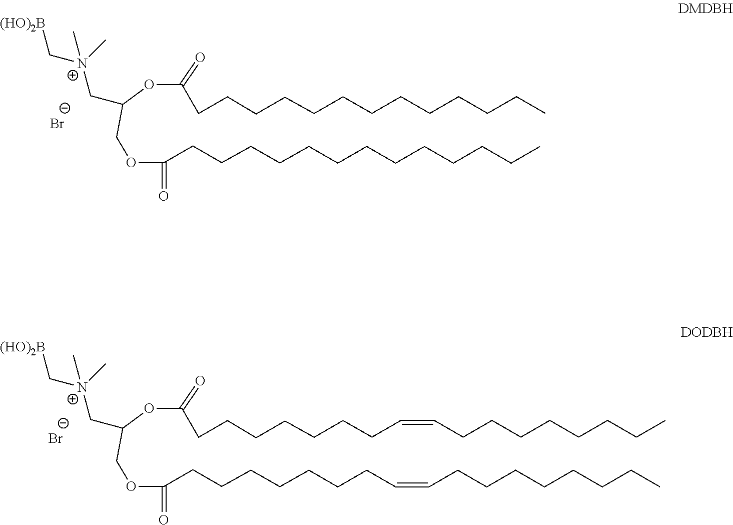 [SMILES: CCCCCCCCCCCCCC(=O)OCC(C[N+](C)(C)CB(O)O)OC(=O)CCCCCCCCCCCCC ; [Br-] ; CCCCCCCC/C=C\CCCCCCCC(=O)OCC(C[N+](C)(C)CB(O)O)OC(=O)CCCCCCC/C=C\CCCCCCCC ; [Br-]]

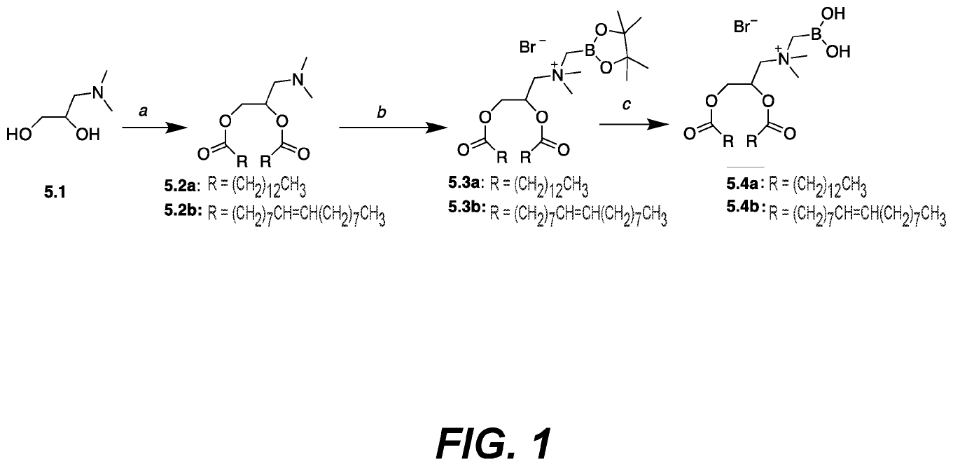

Starting with 3-(dimethylamino)propane 5.1 (FIG. 1), the hydrophobic domain of the boron-containing cationic lipid N-(boronomethyl)-N,N-dimethyl-2,3-bis(tetradecanoyloxy)propan-1-aminium bromide (DMDBH) was added by an acylation procedure using myristoyl chloride. Reaction of the resultant amino bis-ester 5.2a with 2-(bromomethyl)-4,4,5,5-tetramethyl-1,3,2-dioxaborolane delivered the quaternary ammonium bromide salt 5.3a.

For FIG. 1, the conditions of the reactions are: a. myristoyl chloride or oleoyl chloride, TEA, DMAP, CH₂Cl₂, 0° C., rt, 12 h (97%); b. 2 (bromomethyl)-4,4,5,5-tetramethyl-1,3,2-dioxaborolane, CH₂Cl₂, 50° C., sealed tube, 8 h (89%); c. NaOI₄, HCl (2.0 M), H₂O/THF 0° C.-rt, 12 h (71%).

First, 3-(dimethylamino)propane 5.1 was reacted with myristoyl chloride in the presence of DMAP and TEA to generate 5.2a (FIG. 1). The byproduct (Et₃N—HCl) was filtered and the crude ester purified by silica column chromatography using 10% methanol in dichloromethane. The ¹³CNMR of 5.2a shows the characteristic carbonyl carbon of 5.2a at δ 173 ppm. Also evident are the methine proton —CHOC(O)R at δ 5.19 ppm and the methylene protons —CH₂OC(O)R) at δ 4.08 and 4.36 ppm, which resonate downfield relative to the diol starting material.

The bis-ester 5.2a was converted to N,N-dimethyl-2,3-bis(tetradecanoyloxy)-N-((4,4,5,5-tetramethyl-1,3,2-dioxaborolan-2-yl)methyl)propan-1-aminium 5.3a by reaction with 2-(bromomethyl)-4,4,5,5-tetramethyl-1,3,2-dioxaborolane in a pressure tube at 50° C. for 8 h (FIG. 1, Step b). After cooling, the excess solvent was evaporated using a rotary evaporator in a closed fume hood. The crude dioxaborolane 5.3a (FIG. 1) was purified by silica column chromatography, (CH₂Cl₂:CH₃OH, 9:1) to yield 89% of 5.3a as a light-yellow solid. Conspicuously evident is the downfield shift of the N-methyl protons in —CH₂N(CH₃)₂CH₂B— to around δ 3.6 ppm from around δ 2.2 ppm in —CH₂N(CH₃)₂. Further, the methine proton —CHOC(O)R moved even more downfield from δ 5.19 ppm in 5.2a to 5.6 ppm in 5.3a. Also, the methylene protons —CH₂OC(O)R) shifted downfield from δ 4.08 and 4.36 ppm in 5.2a to δ 4.52 and 4.63 ppm in 5.3a.

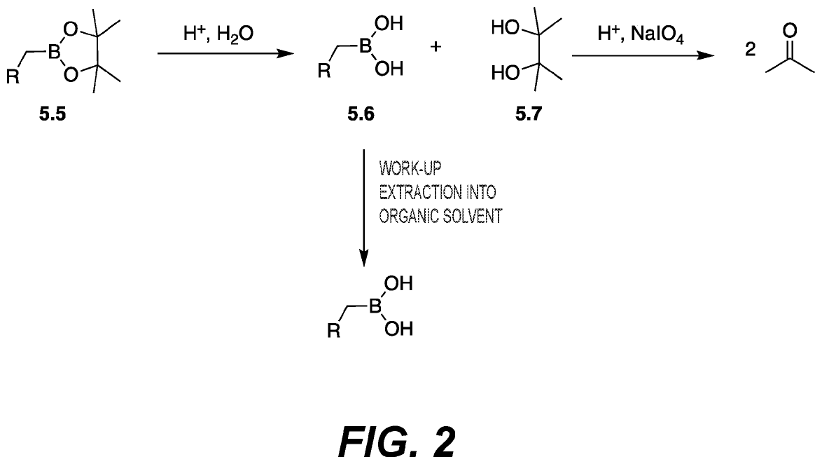

Amine quaternization was followed by hydrolysis of the boronic ester 5.3a in the presence of NaIO₄ to reveal the boronic acid group. Our initial synthesis using this route delivered DMDBH as the bromide salt in 71% yield. The use of sodium periodate in the hydrolysis step is helpful to the success of the deprotection step as it cleaves the vicinal diol 5.7 that is formed on hydrolysis, otherwise boronic acid 5.6 could react with 5.7 on extraction into organic phase and reform the boronic ester 5.5 (FIG. 2). FIG. 2 shows the role of sodium periodate in the boronic ester deprotection step.

To accomplish the hydrolysis, boronic ester 5.3a was dissolved in a H₂O/THF mixture (1:4) and deprotected with aqueous HCl (2.0 M) in the presence of NaIO₄. The crude product was subsequently dried under vacuum for 30 minutes and carefully neutralized by dropwise addition of saturated aqueous NaHCO₃. After extracting the organic portion, the crude boronic acid 5.4a was purified via silica gel column chromatography (CH₂Cl₂:CH₃OH, 9:1) to afford pure 5.4a (FIG. 1, Step c). The protons —CH₂B(OH)₂— shifted to δ 2.80 ppm from δ 3.20 ppm and 3.46 ppm in 5.3a. The ¹¹BNMR referenced to BF₃·OEt₂ in CDCl₃ at δ 0.00 ppm, shows the RB(OH)₂ peak at δ 19.72 ppm.

We also prepared the bis-oleoyloxy analog DODBH, a bis(mono-unsaturated C18) analog, using the same synthetic route. First, the 1,2-diol 5.1 (FIG. 1) was converted to the bis-ester, 3-(dimethylamino)propane-1,2-diyl ditetradecanoate 5.2b (FIG. 1), by reaction with 2.2 eq. of oleoyl chloride (FIG. 1, Step a). The presence of the alkene protons —CH₂CH═CHCH₂— at δ 5.33 ppm in the proton nmr spectrum of 5.2b are a clear indication of the success of the formation of the bis-ester 5.2b. In addition, the methine proton —CHOC(O)R at δ 5.17 ppm in compound 5.2b is similar to the δ 5.19 ppm resonance of this proton in 5.2a.

Bis-ester 5.2b then was converted to the corresponding ammonium bromide salt 5.3b by reaction with 2-(bromomethyl)-4,4,5,5-tetramethyl-1,3,2-dioxaborolane in a pressure tube at 50° C. for 8 h (FIG. 1, Step b). After cooling, the excess solvent was evaporated using a rotary evaporator in a closed fume hood. The crude dioxaborolane 5.3b was purified by silica gel column chromatography (CH₂Cl₂:CH₃OH, 9:1) to yield 89% of 5.3b as a light-yellow solid. ¹HNMR spectra of 5.3a and 5.3b show methylene protons —CH₂OC(O)R in 5.3b around δ 4.49 and S 4.58 ppm, similar to what was observed with 5.3a (S 4.52 and 4.63 ppm).

The ¹HNMR spectrum of the boronic ester δ 5.3b also shows a downfield shift of the methine proton —CHOC(O)R to δ 5.59 ppm from δ 5.17 ppm in compound 5.2b, clearly indicating the formation of the ammonium salt, which provides the electron-withdrawing influence responsible for the downfield shift.

Finally, as in the synthesis of DMDBH, boronic ester 5.3b was dissolved in a H₂O/THF mixture (1:4) and deprotected with aqueous HCl (2.0 M) in the presence of NaIO₄ to afford the bis-oleoyloxy analog DODBH 5.4b.

### Example Set A2—Boronic Compounds—Synthesis and Characterization—Additional Details

### 3-(Dimethylamino)propane-1,2-diyl ditetradecanoate (5.2a)

To an oven-dried round bottom flask, was added 3-(dimethylamino)propane-1,2-diol (0.24 g, 2.0 mmol) and 4-(N,N-dimethylamino)pyridine (0.012, 0.1 mmol). The mixture was dissolved in dry dichloromethane (0.2M). Myristoyl chloride (1.08 g, 4.4 mmol) was subsequently added before the dropwise addition of triethylamine (0.44 g, 4.4 mmol) at 0° C. The reaction mixture turned orange after the addition of triethylamine. The reaction mixture was allowed to warm to room temperature and stirred for 12 h. The resultant suspension was filtered to remove the solid by-product and the filtrate was concentrated by rotary evaporation to afford the crude product. The crude product was purified by silica column chromatography (CH₂Cl₂:MeOH 9:1) to afford 5.2a (1.05 g, 97%) as a light yellow viscous liquid; TLC, R_{f} 0.35, (CH₃OH:CH₂Cl₂ (1:9), p-Anisaldehyde stain); FT-IR 2921, 2852, 1740, 1465, 1166 cm⁻¹; ¹H NMR (500 MHz, CDCl₃) δ 0.87 (t, J=7.0 Hz, 6H); 1.27 (m, 38H), 1.60 (m, 4H); 2.55 (s, 6H); 2.29 (m, 4H); 2.44 (m, 2H); 4.08 (m, 1H); 4.35 (d, J=12 Hz, 1H); 5.19 (m, 1H); ¹³C NMR (125 MHz, CDCl₃) δ14.7, 23.0, 25.3 (2 signals), 29.4, 29.5, 29.6, 29.7, 29.8, 30.0, 32.3, 34.5, 34.8, 46.3, 59.7, 64.2, 69.5, 173.5, 173.8. HRMS m/z calcd [C₃₃H₆₆O₄N]+[M+H]⁺ 540.4986, observed 540.4988.

### 3-(Dimethylamino)propane-1,2-diyl dioleate (5.2b)

To an oven-dried round bottom flask, was added 3-(dimethylamino)propane-1,2-diol (0.59 g, 5.0 mmol) and 4-(N,N-dimethylamino)pyridine (0.030, 0.25 mmol). The mixture was dissolved in dry dichloromethane (0.2M). Oleoyl chloride (3.31 g, 11.0 mmol) was subsequently added before the dropwise addition of triethylamine (1.11 g, 11.0 mmol) at 0° C. The reaction mixture turned orange after the addition of triethylamine. The reaction mixture was allowed to warm to room temperature and stirred 12 h. The resultant suspension was filtered to remove the solid by-product and the filtrate was concentrated by rotary evaporation to afford the crude product. The crude product was purified by silica gel column chromatography (CH₂Cl₂:MeOH 9:1) to afford 5.2b (3.15 g, 97%) as a light yellow viscous liquid; TLC, Rf 0.35, (CH₃OH:CH₂Cl₂ (1:9), p-Anisaldehyde stain); ¹H NMR (500 MHz, CDCl₃) δ 0.87 (t, J=7.0 Hz, 6H); 1.28 (d, J=16 Hz, 42H), 1.59 (m, 4H); 2.01 (m, 8H); 2.27 (s, 6H); 2.29 (m, 4H); 2.46 (m, 2H); 4.08 (q, J=6.5 Hz, 1H); 4.35 (dd, J=3 Hz, 1H); 5.19 (m, 1H), 5.33 (m, 4H); ¹³C NMR (125 MHz, CDCl₃) δ14.0, 22.7, 24.9 (2 signals), 27.1, 27.2, 29.0, 29.1, 29.2, 29.3, 29.5, 29.7, 29.8, 31.9, 34.1, 34.3, 45.8, 59.3, 63.8, 69.0, 129.6, 129.9, 173.0, 173.2. HRMS m/z calcd [C₄₁H₇₈O₄N]+[M+H]⁺: 648.5925, observed 648.5926.

### N,N-Dimethyl-2,3-bis(tetradecanoyloxy)-N-((4,4,5,5-tetramethyl-1,3,2-dioxaborolan2-yl)methyl)propan-1-aminium bromide (5.3a)

A pressure tube was charged with a solution of lipid 5.2a (1.05 g, 1.95 mmol) in CH₂Cl₂ (2.0 mL). To the solution was added 2-(bromomethyl)-4,4,5,5-tetramethyl-1,3,2dioxaborolane (0.516 g, 2.0 mmol). The tube was sealed and then heated at 50° C. for 8 h. On cooling, the tube was opened, the excess solvent was evaporated using a nitrogen stream in a closed fume hood. The residue was purified by silica gel column chromatography (CH₂Cl₂:CH₃OH 9:1) to yield lipid 5.3a (1.32 g, 89%) as a light yellow solid; TLC, Rf 0.42 (CH₃OH:CH₂Cl₂ (1:9), p-Anisaldehyde stain); FT-IR 2984, 1737, 1372, 1233, 1043 cm⁻¹; ¹H NMR (500 MHz, CDCl₃) δ 0.87 (t, J=7.0 Hz, 6H); 1.28 (m, 52H), 1.59 (m, 4H); 2.33 (m, 4H); 3.26 (d, J=16 Hz, 1H); 3.45 (d, J=17 Hz, 1H); 3.60 (d, J=18.5 Hz, 6H); 3.98 (m, 1H); 4.12 (m, 1H); 4.52 (dd, J=3.5, 3.0 Hz, 1H); 4.66 (d, J=14 Hz, 1H); 5.60 (m, 1H); ¹³C NMR (125 MHz, CDCl₃) δ14.2, 22.8, 24.5, 24.7, 24.9, 25.2, 29.2 (2 signals), 29.5, 29.6, 29.8, 32.0, 34.0, 34.3, 54.8, 55.3, 63.4, 65.3, 66.0, 85.9, 173.0, 173.2; ¹¹BNMR (128 MHz, CDCl₃) δ 31.21; HRMS m/z calcd [C₄₀H₇₉O₆NB]⁺ [M+H]⁺: 680.6002, observed 680.5999.

### N,N-Dimethyl-2,3-bis(oleoyloxy)-N-((4,4,5,5-tetramethyl-1,3,2-dioxaborolan-2-yl)methyl)propan-1-aminium bromide (5.3b)

A pressure tube was charged with a solution of lipid 5.2b (2.11 g, 3.26 mmol) in CH₂Cl₂ (2.0 mL). To the solution was added 2-(bromomethyl)-4,4,5,5-tetramethyl-1,3,2dioxaborolane (0.86 g, 3.91 mmol). The tube was sealed and then heated at 50° C. for 8 h. On cooling, the tube was opened, the solvent was evaporated using a nitrogen stream in a closed fume hood. The residue was purified by silica column chromatography (CH₂Cl₂:CH₃OH 9:1) to yield lipid 5.3b (2.40 g, 85%) as a light yellow solid; TLC, R_{f} 0.48 (CH₃OH:CH₂Cl₂ (1:9), p-Anisaldehyde stain); ¹H NMR (500 MHz, CDCl₃) δ 0.86 (t, J=7.0 Hz, 6H); 1.26 (m, 52H), 1.58 (m, 4H); 1.99 (d, J=4 Hz, 8H); 2.32 (m, 4H); 3.24 (d, J=16.5 Hz, 1H); 3.37 (d, J=32 Hz, 1H); 3.59 (d, J=18.5 Hz, 6H); 3.97 (q, J=9 Hz 1H); 4.09 (d, J=5 Hz, 1H); 4.50 (d, J=3 Hz, 1H); 5.31 (m, 4H); 5.59 (bs, 1H); ¹³C NMR (125 MHz, CDCl₃) δ13.9, 22.5, 24.2, 24.4, 24.5, 24.6, 24.8, 24.9, 27.0, 27.1, 28.9, 29.0, 29.1(2 signals), 29.2, 29.4, 29.6, 31.8, 33.7, 34.0, 54.4, 55.1, 63.3, 64.6, 65.9, 85.5, 129.5, 129.8, 172.7, 172.9; ¹¹BNMR (128 MHz, CDCl₃) δ 31.21; HRMS m/z calcd [C₄₈H₉₁O₆NB]⁺ [M+H]⁺: 788.6934, observed 788.6944.

### N-(Boronomethyl)-N,N-dimethyl-2,3-bis(tetradecanoyloxy)propan-1-aminiumbromide (DMDBH) (5.4a)

To an oven-dried round bottom flask was added 5.3a (0.66 g, 0.87 mmol) and sodium periodate (0.557 g, 2.61 mmol). The mixture was dissolved in a H₂O/THF mixture (2 mL, 1:4). The reaction mixture was stirred at 0° C. and 2.0 M aq. HCl (0.5 mL) was added dropwise. The mixture was allowed to warm to room temperature and allowed to stir for 12 h. After completion of the reaction, the reaction mixture was neutralized with sat. NaHCO₃ and stirred at room temperature 15 min before extracting with dichloromethane. The excess dichloromethane was removed by rotary evaporation under reduced pressure to afford the crude product. The crude product was purified by silica gel column chromatography (CH₂Cl₂:MeOH 9:1) to afford 5.4a (0.42 g, 71%) as a light-brown solid; TLC, R_{f} 0.25 (CH₂Cl₂:MeOH 9:1, p-Anisaldehyde stain); FT-IR 3420, 2919, 2851, 1739, 1467 cm⁻¹; ¹H NMR (500 MHz, CD₃OD) δ 0.90 (t, J=7.0 Hz, 6H); 1.29 (m, 38H), 1.63 (m, 4H); 2.36 (m, 4H); 2.80 (bs, 1H); 3.15 (d, J=6.5 Hz, 6H); 3.77 (m, 1H); 4.05 (m, 1H); 4.50 (d, J=3.5, Hz, 1H); 5.63 (m, 1H); ¹³C NMR (125 MHz, CD₃OD) δ14.5, 23.7, 25.8, 25.9, 30.2, (2 signals), 30.4, 30.5, 30.7, 30.8 (2 signals), 33.1, 34.7, 35.1, 54.7, 54.8, 64.6, 66.9, 67.6, 174.0, 174.5; ¹¹BNMR (128 MHz, CDCl₃) δ 19.74; HRMS m/z calcd [C₃₄H₆₉O₆NB]+[M+H]+: 598.5212, observed 598.5211.

### N-(Boronomethyl)-N,N-dimethyl-2,3-bis(oleoyloxy)propan-1-aminium bromide (DODBH) (5.4b)

To an oven-dried round bottom flask, was added 5.3b (0.72 g, 0.91 mmol) and sodium periodate (0.586 g, 2.74 mmol). The mixture was dissolved in a H₂O/THF mixture (2 mL, 1:4). The reaction mixture was stirred at 0° C. and 2.0 M aq. HCl (0.5 mL) was added dropwise. The mixture was allowed to warm to room temperature and allowed to stir for 12 h. After completion of the reaction, the reaction mixture was neutralized with sat. NaHCO₃ and stirred at room temperature 15 min before extracting with dichloromethane. The excess dichloromethane was removed by rotary evaporation under reduced pressure to afford the crude product. The crude product was purified via silica gel column chromatography (CH₂Cl₂:MeOH 9:1) to afford 5.4b (0.50 g, 70%) as a light-brown viscous liquid; TLC, R_{f} 0.32 (CH₂Cl₂:MeOH 9:1, p-Anisaldehyde stain); ¹H NMR (500 MHz, CDCl₃) δ 0.89 (t, J=7.0 Hz, 6H); 1.26 (bs, 42H), 1.61 (m, 4H); 1.96 (m, 4H); 2.34 (m, 4H); 3.32 (d, J=15 Hz, 6H); 3.51 (d, J=6.0 Hz, 1H); 3.66 (s, 2H); 3.73 (d, J=6 Hz, 1H); 3.90 (d, J=14 Hz, 6H); 4.04 (m, 1H); 4.13 (t, J=6 Hz, 6H); 4.43 (m, 1H); 4.51 (t, 1H), 5.38 (s, 4H), 5.55 (bs, 1H)

### Example Set B—Boron Compounds, Lipid Particles, Lipoplexes, and RNA Delivery

This illustrative example shows that addition of lipids containing boronic acid functionality to lipoplexes (i.e., lipid-RNA complexes), such as those derived from hydroxylated cationic lipids, can improve the RNA-delivery activity of the lipoplexes. Without being bound by theory, it is believed that the resultant boronic acid-diol interactions within the liposome bilayers of the lipoplex result in boronate ester formations that covalently link the boronic acid and diol lipids together and strengthen the assembly; the added stability translates to greater intracellular delivery of the RNA payload.

Two representative members of boron compounds (C14 analog DMDBH and C18:1 analog DODBH) have been shown to improve the activity of a hydroxylated oxime ether-based liposomal delivery system in in vitro experiments, as discussed below.

OELs (oxime ether lipids), such as OEL4 and OEL5, contain oxime ether linkages that connect polar, cationic domains to long-chain hydrophobic domains. They can be used as DNA and RNA delivery agents. These cationic amphiphiles can be co-formulated with a neutral phospholipid (dioleoyl-phosphatidylethanolamine (DOPE)) and a PEG-lipid (1,2-distearoyl-sn-glycero-3-phosphoethanolamine-N-[methoxy(polyethylene glycol)-350](DSPE-PEG350)) as liposome suspensions and then mixed with siRNA to form lipoplexes. The OEL-based lipoplexes can efficiently deliver siRNA payloads to cells in vitro and in vivo.

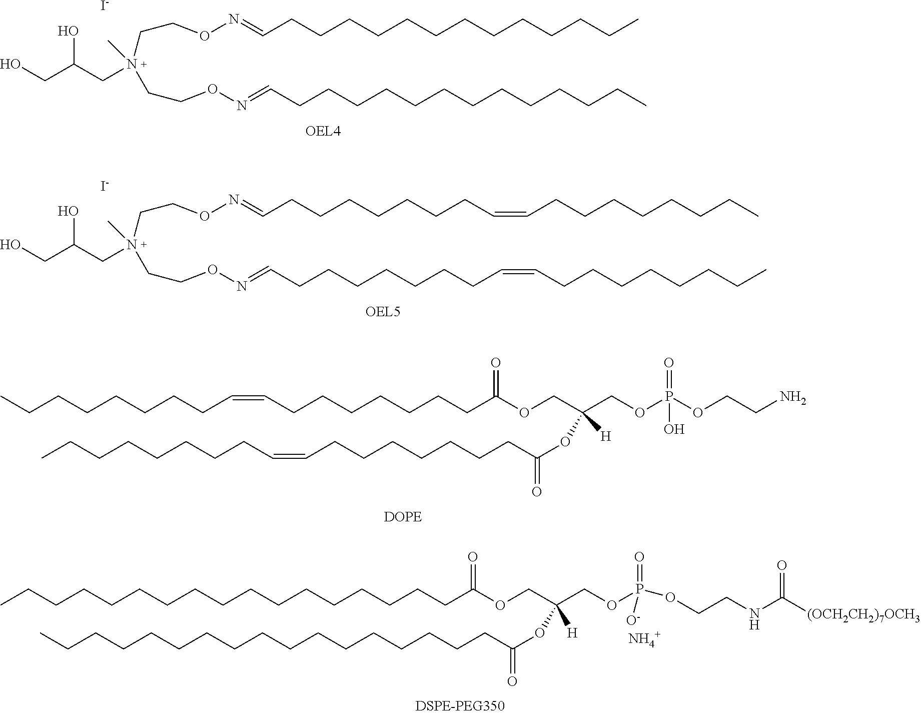

In some instances, DMDBH, when formulated as liposomes with DOPE and DSPE-PEG350, is not an effective siRNA delivery agent. However, when DMDBH is added to OEL4-based siRNA lipoplexes in varying concentrations, the delivery of the siRNA payload to cells is enhanced, as measured by increases in the gene silencing activity.

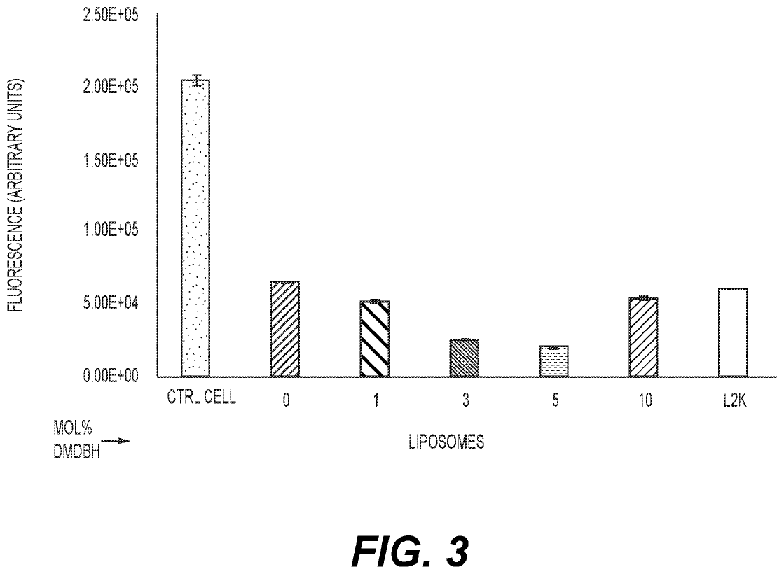

Standard Formulation. The gene silencing activities of OEL4-based anti-eGFP DsiRNA lipoplexes with and without DMDBH were assessed in GFP-expressing human breast adenocarcinoma cells (MDA-MB-231). Equimolar OEL4:DOPE liposomes were prepared at 1 mg/mL in nuclease-free water followed by addition of DMDBH (0-10 mol %) and DSPE-PEG350 (3 mol %). The resultant liposome formulations (180 μL) then were mixed with DsiRNA (1.8 μL of a 10 mM stock solution) to form lipoplexes. Treatment of cells with lipoplexes containing 3 or 5 mol % of DMDBH showed >20% increase in gene silencing relative to the control (0 mol % DMDBH), with overall silencing reaching 88% and 90%, respectively (FIG. 3). In comparison, cells treated with Lipofectamine-based (L2K) lipoplexes (reference transfection agent) showed ˜72% gene silencing.

The anti-eGFP DsiRNA used had the following sequences:

(SEQ ID NO: 1)
Sense (5′ to 3′): ACCCUGAAGUUCAUCUGCACCACCG
(SEQ ID NO: 2)
Antisense (5′ to 3′): CGGUGGUGCAGAUGAACUUCAGGGUCA

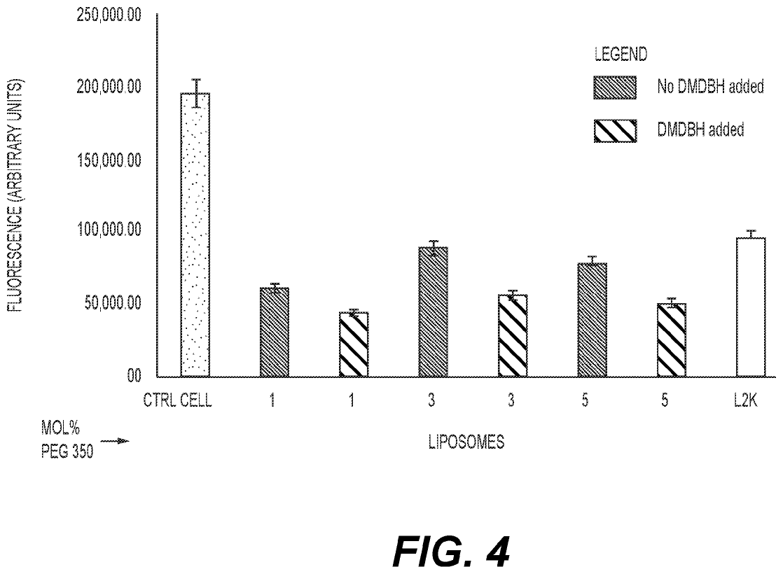

Pre-mix Formulation. To examine whether the order of lipid introduction during lipoplex formulation impacts gene silencing activity, we tested a ‘pre-mix’ formulation protocol in which DMDBH:DOPE (1:1) mixture (90 μL of a 1 mg/mL stock solution) was first mixed with anti-eGFP DsiRNA (1.8 μL of a 10 mM stock solution). After incubation for 30 mins, OEL:DOPE (1:1) liposomes (90 μL of a 1 mg/mL stock solution) containing 1-5 mol % DSPE-PEG350 were added to form the lipoplexes that were then evaluated in GFP-expressing MDA-MB-231 cells (FIG. 4). The gene silencing activities of the lipoplexes prepared using the pre-mix formulation were superior to the control OEL4:DOPE:DSPE-350 lipoplexes at each mol % of DSPE-350 examined, and all DMDBH-containing formulations showed silencing activity greater than the industry standard L2K-based lipoplexes. A silencing enhancement of ˜17% was observed using the DMDBH pre-mix formulation protocol relative to the control OEL4:DOPE:DSPE-350 lipoplex.

Pre-mix Formulation Using PBA. To probe whether addition of a different boronic acid would improve siRNA silencing activity, we substituted phenyl boronic acid (PBA) for DMDBH in a pre-mix experiment. A PBA:DOPE (1:1) mixture (90 μL of a 1 mg/mL stock solution) was first added to anti-eGFP DsiRNA (1.8 μL of a 10 mM stock solution) and incubated for 30 minutes. OEL4:DOPE:DSPE-350(1:1:0.03) liposomes (90 μL of a 1 mg/mL stock solution) then were added to form the PBA-containing lipoplexes.

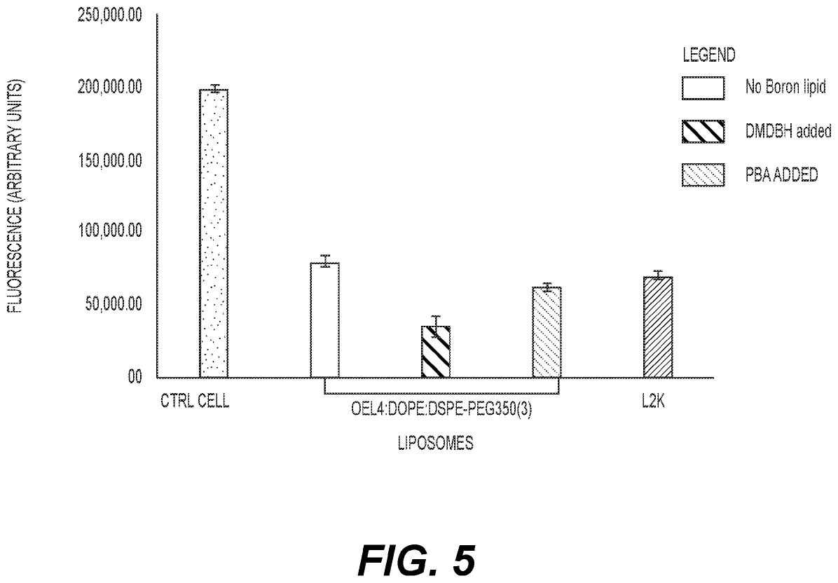

OEL4:DMDBH:DOPE:DSPE-350(1:1:2:0.03) lipoplexes were prepared using the pre-mix formulation protocol for comparison to the PBA experiment. The data (FIG. 5) show that addition of PBA slightly enhances (˜9%) gene silencing relative to the control OEL4:DOPE:DSPE-350(1:1:0.03) lipoplexes. Once again, the DMDBH-containing lipoplexes showed superior activity (>20% improvement, 83% silencing) compared to the OEL-control lipoplexes (60% silencing).

These data demonstrate that addition of DMDBH to OEL4:DOPE:DSPE-PEG350 liposomes, via either standard or pre-mix formulation, improves the gene silencing activity of the derived siRNA lipoplexes.

Finally, we determined the effect of PEGylated DMDBH:OEL4-complexed DsiRNAs on non-specific cellular viability. Various formulations (e.g., varying the mol % of DSPE-PEG350 from 0% to 5%) were incubated with anti-luciferase DsiRNA (anti-luc) duplexes and the resulting lipoplexes were added to human lung cancer cells (A549). All DMDBH:OEL4 formulations tested did not show any non-specific cellular toxicity (data not shown).

### Example Set C—Boronic Compounds and Ester Linkages

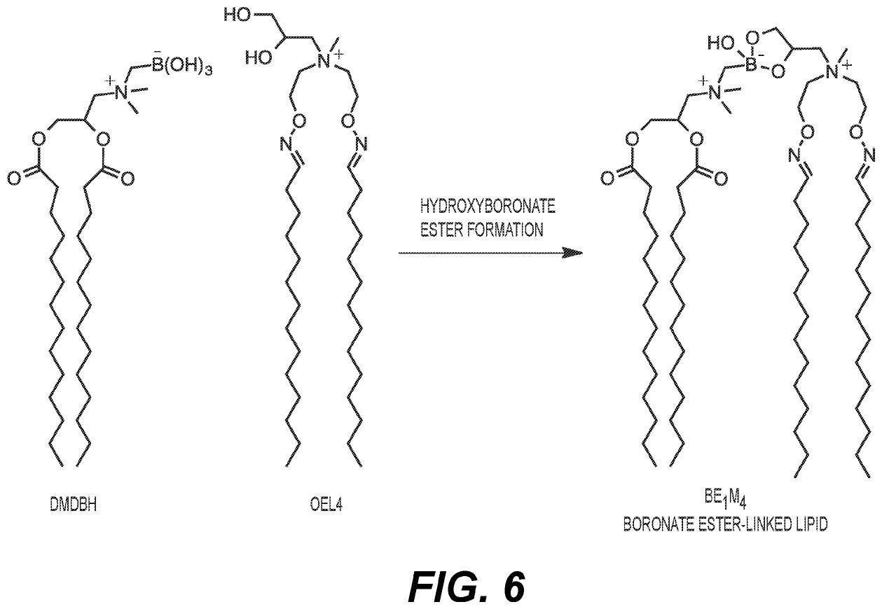

Without being bound by theory, it is possible that the addition of DMDBH enhances overall lipoplex stability through boronate ester linkages formed between adjacent DMDBH and OEL4 lipid molecules in the liposome/lipoplex bilayers (e.g., see FIG. 6). For example, reaction of the boronate acid form of DMDBH with the 1,2-diol moiety of OEL4 might form a hydroxyboronate ester that is stable to lipoplex formulation conditions (neutral pH). Enhanced lipoplex stability appears to translate to improved activity, perhaps due to better protection of the siRNA payload, without being bound by theory. Cleavage of the hydroxyboronate ester linkage is possible when the pH falls to near ˜6, the estimated pH range of DMDBH. Thus, without being bound by theory, stabilized lipoplexes entering cells via an endocytic pathway may gradually release the polynucleotide payload as the linking boronate esters are hydrolyzed on endosome/lysosome acidification (pH ˜5-5.5).

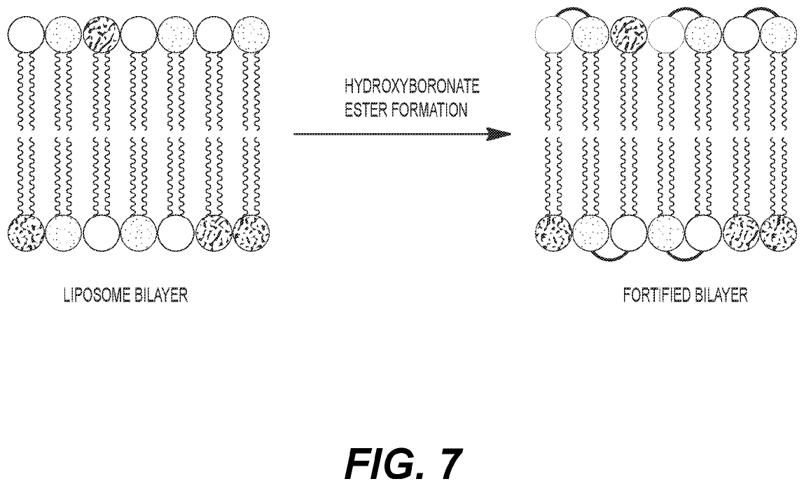

As such, the extent of bilayer fortification by linking together adjacent lipids might be modulated by controlling the DMDBH:OEL4 ratio used for lipoplex formulation (illustrated as an example in FIG. 7).

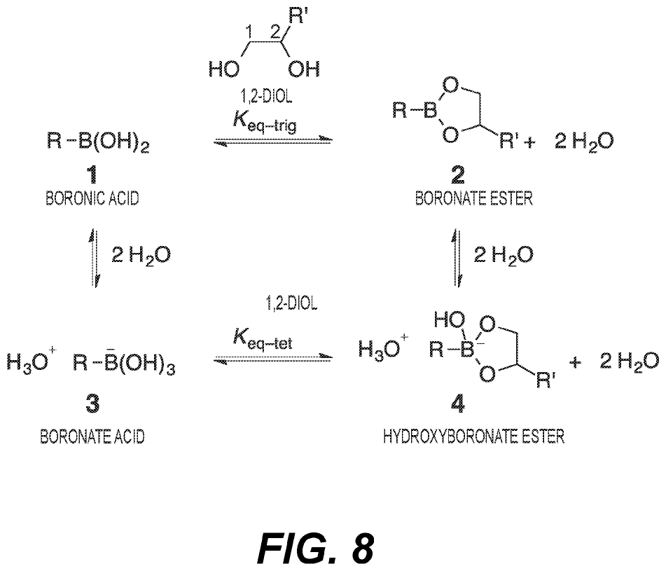

As just discussed, certain embodiments of the invention can involve the formation of a stable hydroxyboronate ester (FIG. 8). In general, boronic acids (1 in FIG. 8) can react reversibly with 1,2-diols (or 1,3-diols) to form cyclic boronate esters, 2. Boronic acids can also react with Lewis bases (e.g., water) to form complexes, such as the tetragonal boronate acids, 3. In general, in aqueous solutions at a pH higher than the pKₐ of the boronic acid, 1, conversion to the charged boronate acids 3 can be favored. Boronate acids 3 also react reversibly with diols to form charged hydroxyboronate esters 4, which can be stable adducts. In contrast, the neutral boronate esters, 2, can be unstable due to the unfavorable ring strain on the sp²-hybridized boron atom, which can sometimes cause rapid equilibrium to either the neutral boronic acids 1 or to the charged hydroxyboronate esters 4. Thus, the formation of stable, charged hydroxyboronate esters 4 can sometimes require the presence of a certain fraction of charged boronate acids 3. In certain instances, this means that when the pKₐ of a boronic acid, 1, is too high, not enough boronate acid, 3, is present at physiological conditions to form the stable ester, 4, and to shift the boronic-acid equilibrium to the charged side in presence of diols. In some embodiments, the boron compounds of the present invention are structurally designed to have a low pKₐ to facilitate favorable equilibrium with diols and form stable hydroxyboronate esters.

As noted in the discussion of FIG. 8, boronate ester formation can be dependent on the pKₐ of both boronic acid and 1,2-diol reactants as well as solution pH. In some embodiments, the present invention exploits the electron-withdrawing influence of the 0-ammonium ion in DMDBH to sufficiently lower the pKₐ of the alkyl boronic acid moiety (pKₐ typically ˜10) to a pKₐ estimated at ˜7, which can facilitate formation of the charged boronate acid under neutral conditions. In certain embodiments, the most acidic boronic acids possess the most electrophilic boron atom that can best form and stabilize a hydroxyboronate anion.

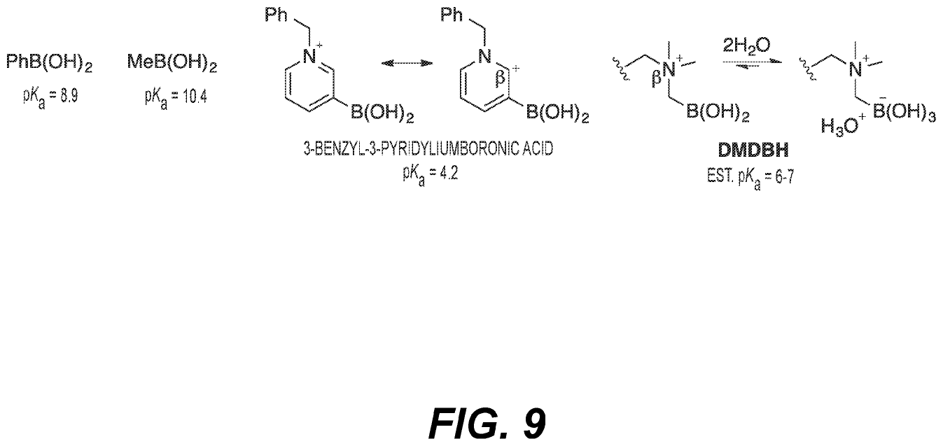

The difference in pKₐ between phenyl boronic acid (pKₐ 8.9) and 3-benzyl-3-pyridylium boronic acid (pKₐ 4.2) (FIG. 9) is near 5 pKₐ units, a result of the strong electron-withdrawing influence of the Q-cation in the pyridylium boronic acid. By analogy, we estimate a similar difference in acidity between an alkyl boronic acid, such as methyl boronic acid (pKₐ 10.4), and DMDBH, which also has a significant β-cation influence. It is thus reasonable to estimate the pKₐ of DMDBH to be sufficiently low (below 7) to favor equilibration to the charged boronate acid form.

Although simple alcohols have pKₐ values in the range of 16, vicinal diols can be more acidic; for example, the pKₐ of 1, 2, 3-trihydroxypropane is 14.4. Given that OEL4 has a 0-ammonium ion, the diol of OEL4 likely has a pKₐ closer to 12, such as that of glucose and fructose, where inductive electron-withdrawing effects are also operative. Acidic diols typically react faster to form boronate esters. The complexation of DMDBH by OEL4 appears to involve reactants that are favorably disposed to form a hydroxyboronate ester.

Experimental evidence for the charged boronate acid form of DMDBH (as depicted in FIG. 6) is provided by zeta-potential measurements on DMDBH liposomes and by comparing these measurements to the zeta-potential measurements on OEL4 liposomes. Both lipids possess tetraalkylammonium ions in their polar domains that impart significant positive zeta-potential values to liposomes derived from these lipids. DMDBH, however, in its charged, tetrahedral boronate acid form would contain an anionic boron that would neutralize the ammonium ion charge. Neutralization in this manner should be obvious from zeta-potential measurements. Zeta-potential measurements on PEGylated DMDBH-derived liposomes show significant reduction in charge relative to PEGylated OEL4-derived liposomes (Table C1). The cationic OEL4 liposomes average ˜33 mV whereas DMDBH liposomes are on average ˜20 mV less charged than OEL4 liposomes. These measurements indicate that the boronic acid group of DMDBH is highly Lewis acidic and readily equilibrates with water to form a significant fraction of the tetrahedral, charged boronate acid, which exerts a neutralization effect on the overall lipid charge.

TABLE C1
Comparison of zeta (ζ)-potentials for DMDBH
and OEL4, each formulated as liposomes with
equimolar DOPE and DSPE-PEG350 (1-5 mol %).

| DMDBH Liposomes | | OEL4 Liposomes | |
|---|---|---|---|
| Liposome | ζ-potential | Liposome | ζ-potential |
| formulation | (mV) | formulation | (mV) |
| DMDBH:DSPE-350(0) | 8.08 | OEL4:DSPE-350(0) | 33.0 |
| DMDBH:DSPE-350(1) | 12.0 | OEL4:DSPE-350(1) | 32.8 |
| DMDBH:DSPE-350(3) | 11.4 | OEL4:DSPE-350(3) | 34.7 |
| DMDBH:DSPE-350(5) | 10.9 | OEL4:DSPE-350(5) | 34.7 |

Evidence to support lipid-lipid interactions between DMBDH and OEL4 is provided by particle size measurements on liposome formulations comprised of these lipids. Boronate ester formation between neighboring lipids in a bilayer (or potentially across bilayers in these multilamellar vesicles) effectively increases the lipid-lipid packing within the liposome, which can be expected to reduce overall particle size. In agreement with this expectation, hydrodynamic size measurements on PEGylated OEL4:DOPE (1:1) liposomes show that gradual increases in the mol % of added DMDBH results in smaller and smaller particles (Table C2).

TABLE C2
Hydrodynamic sizes of PEGylated OEL4:DMDBH:DOPE:DSPE-
350 (1:0.0 to 0.1:1:0.03) liposomes.

| OEL4:DOPE(1:1):DSPE-350(3) + | Size |
|---|---|
| varying mol % DMDBH | diameter (nm) |
| no DMDBH added | 139.6 |
| 1 mol % DMDBH | 102.4 |
| 3 mol % DMDBH | 97.4 |
| 5 mol % DMDBH | 69.9 |
| 10 mol % DMDBH | 66.9 |

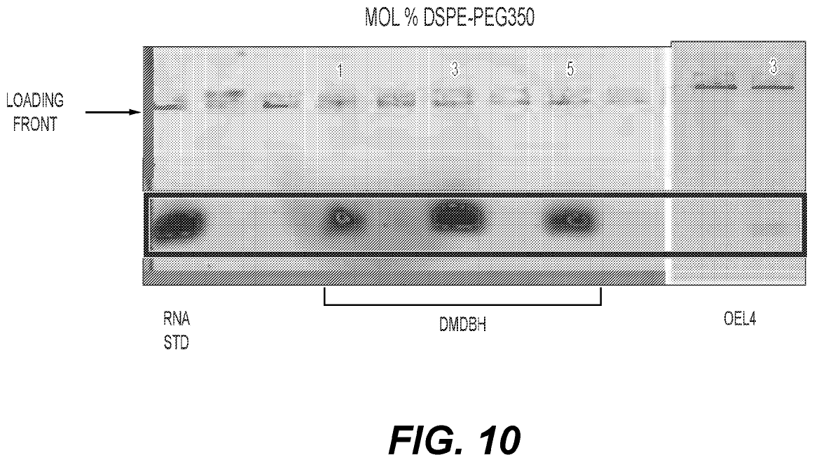

Additional evidence that DMDBH largely exists in its charged boronate acid form, which predisposes it to forming stable hydroxyboronate esters, is provided by RNA-binding studies (FIG. 10). PEGylated liposomes of DMDBH fail to complex labelled RNA due to the anionic charge of the tetrahedral boron effectively neutralizing the ammonium ion; no ion pairing between lipid and RNA-phosphate occurs, so no RNA packaging occurs. In contrast, PEGylated liposomes of cationic OEL4 efficiently bind RNA at the same lipid:RNA ratio.

Preliminary studies using ¹H NMR analysis and high resolution mass spectroscopic analysis of a reaction of DMDBH and OEL4 in organic solvent show evidence of BE₁M₄ linked lipid formation. The preliminary ¹H NMR study show a downfield shift of the indicated protons of OEL4 in the dimer (around δ 5.61 ppm) compared with those of the OEL4 (around δ 3.99 and δ 4.49 ppm), which is consistent with boronic ester formation. The preliminary high resolution mass spectroscopic show a molecular ion was observed (m/z calcd for C₇₀H₁₄₀BN₄O₉⁺ 1192.0708; found, 1192.0753), which is consistent with boronic ester formation.

In some embodiments, the alkyl boronic acid group of the boron compound can be activated by the presence of a β-ammonium ion. This structural feature appears to enable boronate ester formation under physiologic conditions, which can be used for creating reversible, pH-sensitive links between neighboring lipids, not for targeting cellular carbohydrates. For example, when DMDBH is mixed with a transfection-active lipid that possesses a 1,2-diol group (OEL4), boronate ester formation appears to occur and would covalently link the lipids (e.g., BE₁M₄ appears to be formed). The apparent formation of boronate ester-linked lipids within lipoplexes (lipid-siRNA complexes) derived from appropriately functional other lipids (e.g., lipids with 1,3-diol groups or 1,2-diol groups, such as OEL4) improves the gene-silencing activity of the lipoplexes. Further, without being bound by theory, we speculate that the enhanced lipoplex stability afforded by lipid crosslinking contributes to a greater fraction of siRNA delivered into cells. This represents a new, flexible means of enhancing lipoplex performance (e.g., through simple mixing operations).

### Example Set D—Bis(Ammonium Boronic Acid)

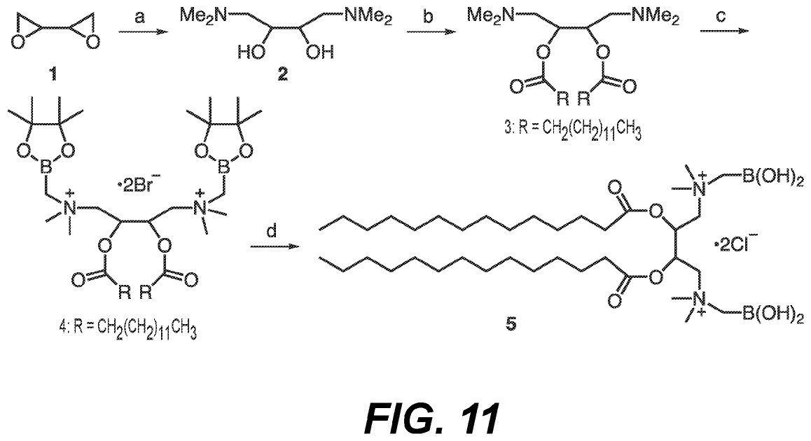

The following example shows how the α-ammonium boronic acid functionality can be incorporated more than once into the polar domain of a lipid. Below are given details for synthesis (FIG. 11) and characterization of a representative bis(ammonium boronic acid) lipid possessing a C₁₄ hydrophobic domain.

### Experimental Procedures

1,4-Bis(dimethylamino)butane-2,3-diol (2). Dimethylamine (40% solution in H₂O, 2.00 mL, 39.5 mmol) was added to a pressure tube containing a magnetic spin bar and cooled to 0° C. followed by slow addition of 1,3-butadiene diepoxide (1) (0.50 mL, 6.46 mmol) dropwise. To this solution, a second, equal portion of dimethylamine (40% solution in H₂O, 2.00 mL, 39.5 mmol) was added dropwise. The tube was sealed and the reaction was stirred at 0° C. for 1 h and then stirred 2 days at room temperature. The tube was opened and the reaction solution was concentrated by rotary evaporation to obtain the crude product which was directly used in the next step without purification; ¹H NMR (400 MHz, CD₃OD) δ 3.67 (m, 1H), 2.47 (m, 2H) 2.29 (s, 6H).

1,4-Bis(dimethylamino)butane-2,3-diyl ditetradecanoate (3). Diol 2 (1.15 g, 6.53 mmol) was added to 2,4-dimethylpyridine (0.04 g, 0.33 mmol) in dry CH₂Cl₂ and the mixture was cooled to 0° C. To this solution was added myristoyl chloride (3.90 mL, 14.3 mmol) dropwise followed by addition of triethylamine (1.98 mL, 14.3 mmol). The reaction mixture was stirred at 0° C. for 1 h and then stirred 17 h at room temperature. The resultant precipitate was removed by filtering the reaction solution using a Buchner funnel and washing the retentate with CH₂Cl₂. The combined filtrate was concentrated by rotary evaporation and the residue was purified using normal-phase column chromatography, eluting with CH₂Cl₂:MeOH (9:1), to afford diester 3 (3.19 g, 83%) as a yellow solid, m.p.=119-121° C.; ¹H NMR (400 MHz, CD₃Cl) δ 5.22 (m, 1H), 2.46 (dd, J=5.5, 6.4 Hz, 1H), 2.38-2.28 (m, 3H), 2.25 (s, 6H), 1.62 (m, 2H), 1.27 (m, 20H), 0.87 (t, J=6.0 Hz, 3H) ppm; ¹³C NMR (100 MHz, CD₃Cl) δ 173.0, 69.8, 59.4, 45.6, 34.2, 31.8, 29.4, 24.9, 22.6, 14.0 ppm; FT-IR 1733 (C═O), 1148 cm⁻¹; HRMS, m/z calculated C₃₆H₇₄N₂O₄²⁺ 299.2819, found 299.2818.

N¹,N¹,N⁴,N⁴-tetramethyl-2,3-bis(tetradecanoyloxy)-N¹,N⁴-bis((4,4,5,5-tetramethyl-1,3,2-dioxaborolan-2-yl)methyl)butane-1,4-diaminium (4). To a solution of diester 3 (1.27 g, 2.13 mmol) in CH₂Cl₂ (4.00 mL) in a pressure tube was added 2-(bromomethyl)-4,4,5,5-tetramethyl-1,3,2-dioxaborolane pinacol ester (1.90 mL, 10.65 mmol) dropwise. The tube was sealed and the reaction mixture was heated at 50° C. for 24 h. After cooling to rt, the tube was opened and the reaction solution was concentrated using rotary evaporation. The residue was triturated with n-pentane to remove unreacted starting material and dried to afford the product bis(ammonium) dibromide 4 (1.10 g, 50%) as a white solid, m.p.=136-139° C.; ¹H NMR (400 MHz, CD₃OD) δ 5.65 (apparent d, J=7.2 Hz, 1H), 4.06 (dd, J=14.8, 7.6 Hz, 1H), 3.95 (dd, J=14.0, 1.0 Hz, 1H), 3.34 (s, 2H), 3.28 (s, 3H), 3.25 (s, 3H), 2.51 (m, 2H), 1.67 (m, 2H), 1.36-1.29 (m, 32H), 0.90 (t, J=6.4 Hz, 3H) ppm; ¹³C NMR (100 MHz, CD₃OD) δ 173.8, 87.2, 75.8, 68.1, 66.8, 55.2, 34.9, 33.1, 30.6, 25.3, 23.7, 14.4 ppm; ¹¹B NMR (128 MHz, CD₃OD) δ 25.5 ppm; HRMS, m/z calculated C₅₀H₁₀₀B₂N₂O₈²⁺ 439.3828; found 439.3826.

N¹,N⁴-bis(boronomethyl)-N¹,N¹,N⁴,N⁴-tetramethyl-2,3-bis(tetradecanoyloxy)butane-1,4-diaminium (5). Bis(ammonium) dibromide 4 (0.93 g, 0.90 mmol) was dissolved in MeOH (2.00 mL) and the mixture was cooled to 0° C. 0.1 N HCl in MeOH was added dropwise until the pH of the solution reached 2. The reaction solution then was purged with argon for 15 min followed by addition of methylboronic acid (0.32 g, 5.38 mmol) in one portion. The reaction mixture was stirred at 0° C. for 1 h and then stirred overnight at room temperature. The reaction solution was concentrated using rotary evaporation. To the crude product was added CH₃CN (5 mL) followed by sonication for 2 min to enhance dissolution of the volatile byproduct and the unreacted methylboronic acid. The solvent then was removed by rotary evaporation. This step was repeated five times to yield bis(boronic acid) 5 (0.70 g, 90%) as a pale yellow solid, m.p.=134-136° C.; ¹H NMR (400 MHz, CD₃OD) δ 5.65 (m, 1H), 4.10-3.95 (m, 2H), 3.34 (s, 2H), 3.29 (s, 3H), 3.26 (s, 3H), 2.51 (m, 2H), 1.67 (m, 2H), 1.35-1.29 (m, 20H), 0.89 (t, J=6.0 Hz, 3H) ppm; ¹³C NMR (100 MHz, CD₃OD) δ 173.8, 68.1, 66.1, 55.3, 55.0, 34.8, 33.1, 30.6, 25.5, 23.7, 14.5 ppm; ¹¹B (128 MHz, CD₃OD) δ 25.9 ppm; HRMS, m/z calculated C₃₈H₈₀B₂N₂O₈²⁺ calculated 357.3045; found 357.3045.

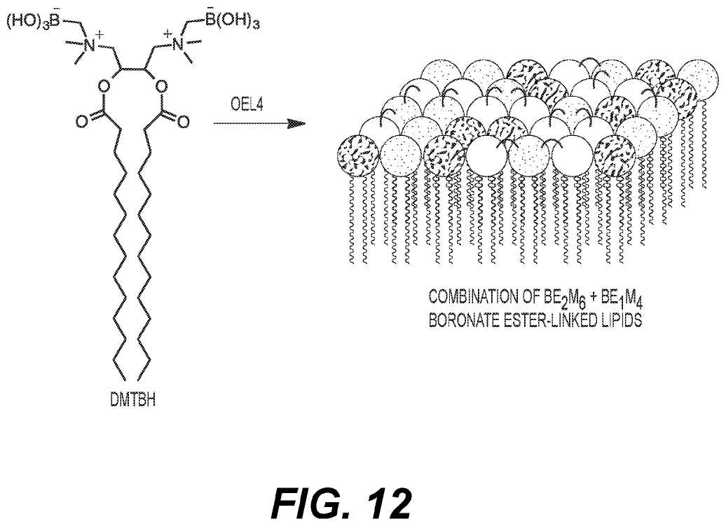

With the knowledge that boron compounds (e.g., DMBDH) can be used to bind 1,2-diol lipids, extrapolation of this concept to the creation of a bis(boronic acid) lipid, such as DMTBH (FIG. 12), would allow even further control in stabilization of liposome/lipoplex formulations. In prophetic example, a bis(1,2-diol) lipid can be envisioned to react with DMTBH to create lipid polymers (BEₓM₍₂ₓ₊₂)) via step growth polymerization, which in turn could improve the outcome of lipoplex-mediated gene therapy.

FIG. 12 provides a representation of reversible boronate ester crosslinking (lines connecting lipid heads) in a lipoplex bilayer leaflet when bis(boronic acid) DMTBH reacts with adjacent OEL4 lipids to form both mono- and bis-linked products.

### Example Set E—Influence of an α-Ammonium Boronic Acid Lipid on Transfection Activity

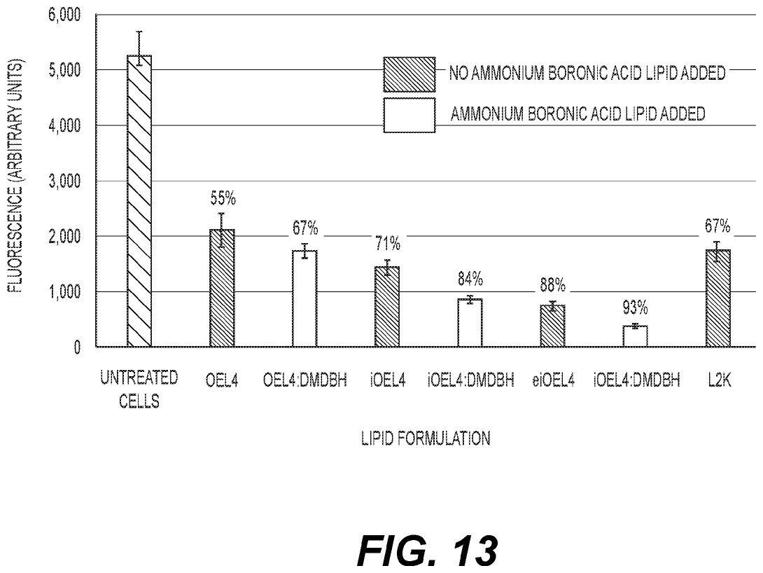

Addition of an α-ammonium boronic acid lipid (DMDBH) to a liposome-siRNA formulation comprised of a 1,2-diol-containing transfection lipid, a PEG-containing lipid, a phospholipid (DOPE), and anti-eGFP siRNA improves the silencing activity of the formulation. Below are the results of adding DMDBH to three different transfection lipid formulations made from three 1,2-diol-containing lipids (below). In each case, addition of the α-ammonium boronic acid lipid improved overall activity (FIG. 13). DMDBH improved the activity of both cationic (OEL4) and ionizable (iOEL4, eiOEL4) transfection lipids.

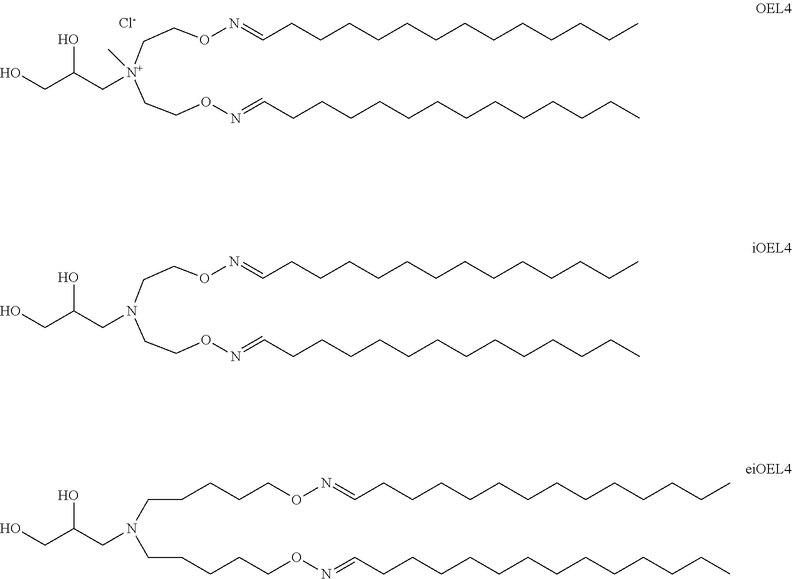 [SMILES: [Cl-] ; CCCCCCCCCCCCC/C=N/OCC[N+](C)(CCO/N=C/CCCCCCCCCCCCC)CC(O)CO ; CCCCCCCCCCCCC/C=N/OCCN(CCO/N=C/CCCCCCCCCCCCC)CC(O)CO ; CCCCCCCCCCCCC/C=N/OCCCCCN(CCCCCO/N=C/CCCCCCCCCCCCC)CC(O)CO]

FIG. 13 shows gene silencing activities of transfection lipid-DsiRNA liposome formulations with and without DMDBH. Liposomes were prepared and then mixed with anti-eGFP DsiRNA. The % eGFP silencing was determined relative to control cells (GFP-expressing MDA-MB-231 cells).

### Experimental Details for Transfection Study

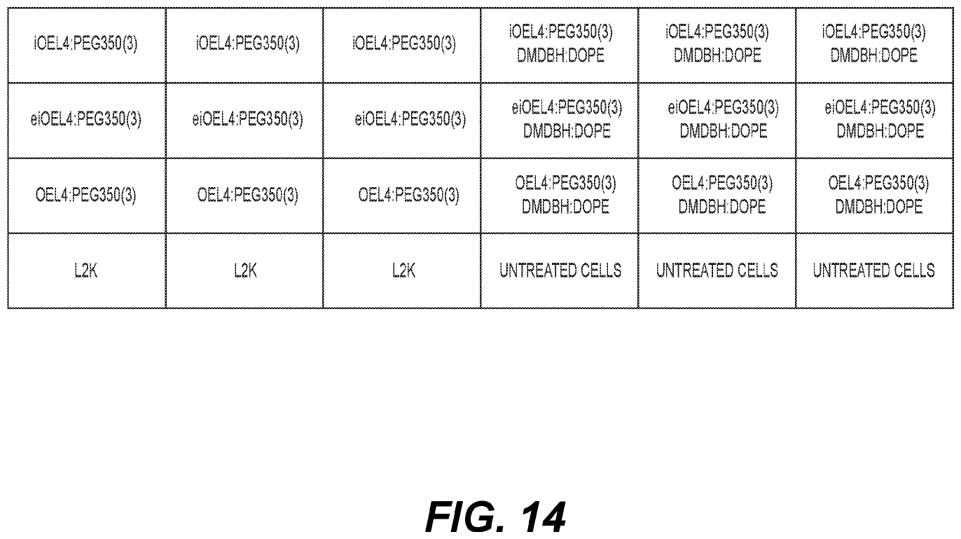

A. Study design. FIG. 14 depicts the experiments performed to determine the effect of added α-ammonium boronic acid lipid DMDBH. FIG. 14 shows the 24-Well plate study design. The number of cells plated on the 24-well plate was ca. 60,000 per well. Cells were plated overnight prior to the day of experiment. In this FIG. 14 table, “PEG350(3)” is 3 mol % of 1,2-distearoyl-sn-glycero-3-phosphoethanolamine-N-[methoxy(polyethylene glycol)-350](ammonium salt), which was added to each liposome formulation. “iOEL4” and “eiOEL4” liposomes were prepared in pH 4 acetate buffer. “OEL4” and “DMDBH” liposomes were prepared in pH 7 nuclease free water. All liposomes contain equimolar amount of DOPE relative to the transfection lipid or DMDBH. “L2K” is Lipofectamine-2000.

B. Liposome formulation. Lipid concentration in all OEL and DMDBH stock liposome formulations: 1 mg/mL total lipid concentration.

1. Add 135 μL of DMDBH:DOPE (1:1) liposomes into three differently labelled 1.5 mL Eppendorf tubes.

2. Add 6 μL of 10 μM stock solution of anti-eGFP siRNA duplex to each tube containing the DMDBH:DOPE liposomes, vortex to mix well and incubate at room temperature for 30 minutes.

3. To each of the 1.5 mL Eppendorf tubes containing the DMDBH:DOPE/siRNA complex (lipoplex), add 135 μL of either OEL4:DOPE:DSPE-PEG350 (1:1:0.03), iOEL4:DOPE:DSPE-PEG350 (1:1:0.03), or eiOEL4:DOPE:DSPE-PEG350 (1:1:0.03) liposomes.

4. Mix 270 μL of either OEL4, iOEL4 or eiOEL4 liposomes and 6 μL of 10 μM stock solution of anti-eGFP siRNA duplex in three separate 1.5 mL Eppendorf tubes, each for formulations without boron lipid. Vortex the lipoplex to mix well.

To another 1.5 mL Eppendorf tube, add 30 μL of Lipofectamine-2000 and 6 μL of 10 μM stock solution of anti-eGFP siRNA duplex, vortex to mix well.

To all the lipoplexes prepared, add FBS-containing DMEM media to a total volume of 1500 μL, then vortex to mix well.

### C. Procedure for Silencing Studies Using MDA-MB-231 GFP Cells.

1. Remove media from the wells.

2. Add 500 μL of the liposome-RNA formulation to the respective well.

3. Incubate for 4-6 hours, remove the media and replace with new media.

4. Incubate at 37° C. for 72 hours.

5. After 72 hours, discard the media and wash the cells with 1×PBS to remove any detached (possibly dead) cells.

6. Trypsinize cells at 37° C. to detach live cells from the bottom of the wells.

7. Add 1 mL DMEM media to each well (formulations were tested in triplicate), pipette back and forth and into a labelled 1.5 mL Eppendorf tube.

8. Centrifuge and discard the media; resuspend the cells in 500 μL PBS and measure the fluorescence using flow cytometry.

### Example Set F—Additional Embodiments

In these examples, LNP is lipid nanoparticles and SM-102 is

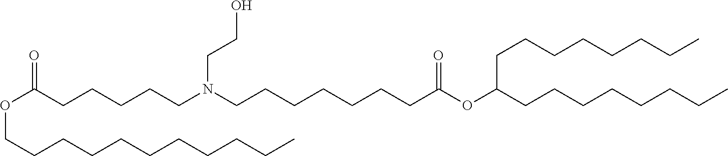 [SMILES: CCCCCCCCCCCOC(=O)CCCCCN(CCO)CCCCCCCC(=O)OC(CCCCCCCC)CCCCCCCC]

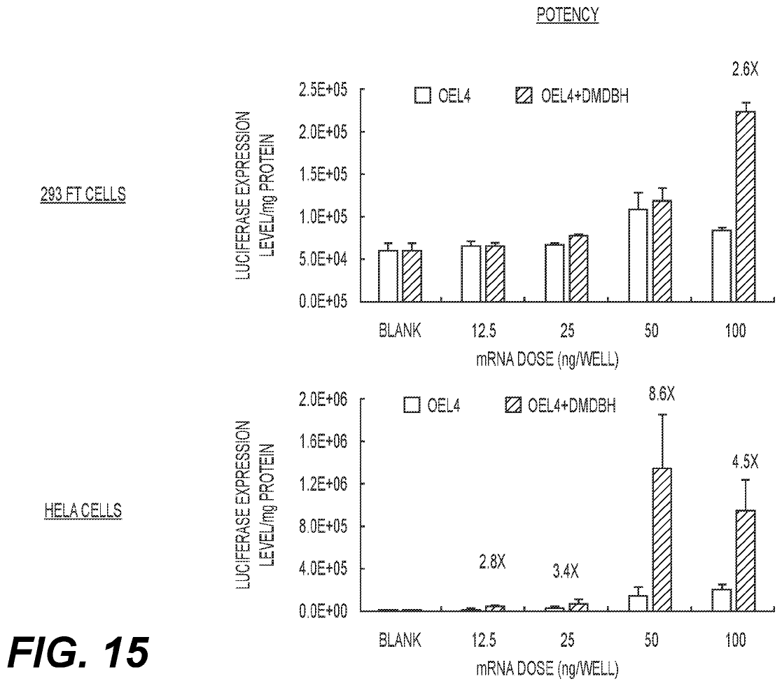

FIG. 15 shows transfection activity of OEL4:DOPE:DSPE-PEG350 liposomes without and with boron lipid DMDBH at different Luc mRNA doses. The OEL4/mRNA complexes were prepared as described below using an N/P ratio=6. The data show that addition of boron lipid to the transfection formulation enhances transfection activity at the higher mRNA doses.

### Materials

Solution without DMDBH (Solution A): OEL4:DOPE:DSPE-PEG350 (molar ratios=1:1:0.03). Total lipid concentration is 1 mg/mL in water.

Solution with DMDBH (Solution D): DMDBH:DOPE (1:1). Total lipid concentration is 1 mg/mL in water.

### Abbreviations:

OEL4 is 2,3-dihydroxy-N-methyl-N,N-bis(2-((((E)-tetradecylidene)amino)oxy) ethyl)propan-1-ammonium iodide;
DOPE is 1,2-dioleoyl-sn-glycero-3-phosphoethanolamine;
DSPE-PEG350 is 1,2-distearoyl-sn-glycero-3-phosphoethanolamine-N-[carboxy(polyethylene glycol)-MW350];
DMDBH is N-(boronomethyl)-N,N-dimethyl-2,3-bis(tetradecanoyloxy)propan-1-aminium bromide;
Luc mRNA is firefly luciferase encoding mRNA.
   Representative Transfection Procedure Using OEL4:DOPE:DSPE-PEG350 (without DMDBH)

Mix 9 μL of Solution A with 66 μL of water. Mix 1.2 μL Luc mRNA (1.24 mg/mL) with 75 μL of water. Add mRNA solution to diluted liposome solution A and vortex mix. Incubate for 30 min. Add 10 μL of resultant complex solution to each well (100 ng mRNA/well).

### Representative Transfection Procedure Using OEL4:DOPE:DSPE-PEG350+ DMDBH:DOPE

Mix 9 μL of Solution D with 66 μL of water. Mix 1.2 μL Luc mRNA (1.24 mg/mL) with 75 μL of water. Add mRNA solution to diluted liposome solution D and vortex mix. Incubate for 30 min. Mix 9 μL of Solution A with 66 μL of water. Add diluted Solution A to the mixture of Solution D with mRNA. Add 15 μL of resultant complex solution to each well (100 ng mRNA/well).

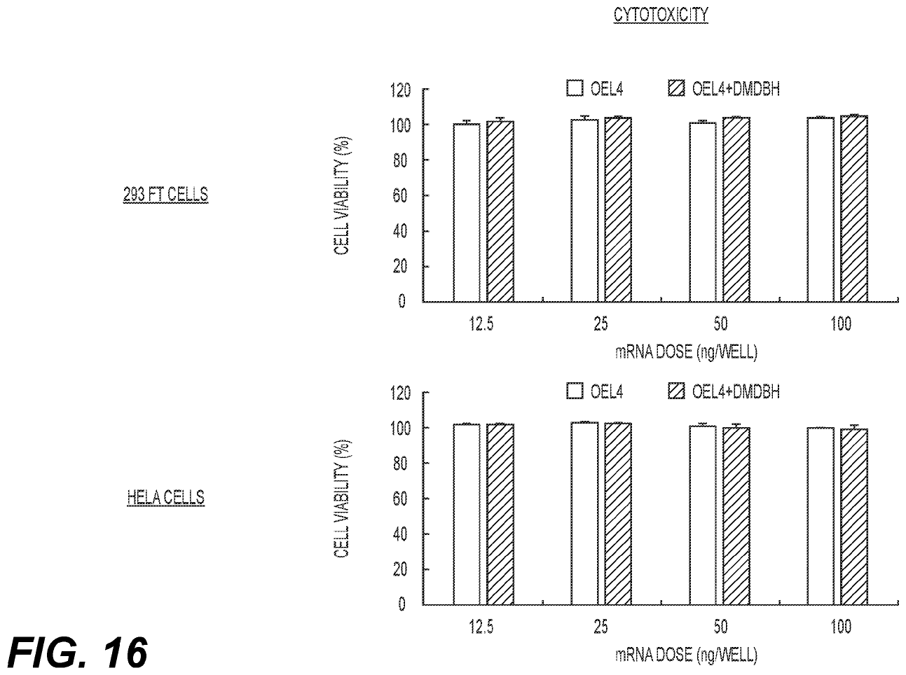

FIG. 16 shows cell viability assay to accompany the transfection experiments depicted in FIG. 15. The results indicate that addition of boron lipid DMDBH to the OEL4/mRNA formulations does not increase associated cytotoxicity. Top graph=results in 293FT cells, bottom graph=results in HeLa cells.

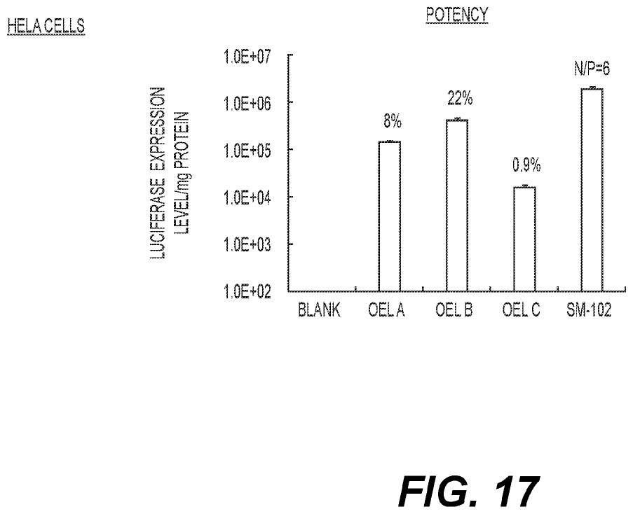

FIG. 17 shows transfection activity of OEL/Luc mRNA formulations relative to standard lipid formulation SM-102 in HeLa cells. All experiments were conducted at an mRNA dose=100 ng/well. OEL transfections were performed at an N/P ratio=64. The SM-102 transfection was performed at an N/P ratio=6.

OEL lipid compositions are as follows:
OEL A=OEL4:DOPE:DSPE-PEG350 (molar ratios 1:1:0.03);
OEL B=OEL5:DOPE:DSPE-PEG350 (1:1:0.03);
OEL C=OEL4:DOPE:DMDBH:DSPE-PEG350 (1:1:1:0.03).

### Abbreviations:

OELS is 2,3-dihydroxy-N-methyl-N,N-bis(2-((((1E,9Z)-octadec-9-en-1-ylidene)amino)oxy)ethyl)propan-1-aminium iodide;
SM-102 is ionizable amino lipid 9-heptadecanyl 8-{(2-hydroxyethyl)[6-oxo-6-(undecyloxy)hexyl]amino}octanoate.

OEL liposomes were formulated as previously described.

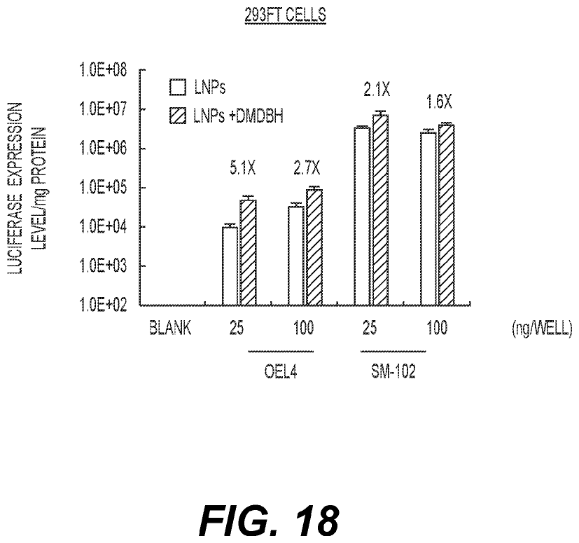

These results show that the formulation containing the boron lipid DMDBH (OEL C), when mixed with the transfection lipid OEL4 prior to complexation with mRNA, is not as effective as the reference SM-102 formulation. Mixing the boron lipid with mRNA before adding the transfection lipid formulation is generally superior to the formulation method used above (see FIG. 18 below).

FIG. 18 shows the influence of mRNA dose (25-100 ng/well) on transfection activity of LNP/Luc mRNA complexes formed using OEL4:DOPE:DSPE-PEG350 (1:1:0.03) liposomes and the industry reference SM-102 formulation without (open bars) and with (hash bars) boron lipid DMDBH in 293FT cells. The OEL/mRNA complex was prepared using OEL4 as previously described at an N/P ratio=12. The SM-102/mRNA experiments were performed at an N/P ratio=6. For experiments in which DMDBH was added (hash bars), the DMDBH:DOPE solution was added to the Luc mRNA prior to addition of the transfection lipid formulation as previously described.

The results show that boron lipid DMDBH enhanced the potency of the OEL4 and SM-102 formulations at both mRNA doses examined.

### Example Set G—Additional Embodiments

1. A boron compound selected from Formula (I) (i.e., Formula (I) is (Ia) and (Ib)), salts of Formula (I), optical isomers of Formula (I), geometric isomers of Formula (I), salts of optical isomers of Formula (I), salts of geometric isomers of Formula (I), and derivatives thereof,

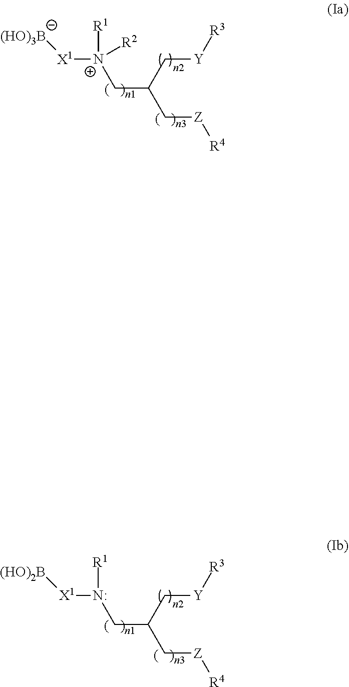

wherein
X is —C(R^{a})(R^{b})—, —C(R^{a})(R^{b})—C(R^{c})(R^{d})—, or —C(R^{a})(R^{b})—C(R^{c})(R^{d})—C(R^{e})(R^{f})—;
R^{a}, R^{b}, R^{c}, R^{d}, R^{e}, and R^{f} can be the same or different and are H, halogen (e.g., F, Cl, Br, or I), —CN, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, or —OCF₃;
R¹ and R² can be the same or different and are H, hydroxy (—OH), methanoyl (—COH), nitro (—NO₂), —NH₂, —N(CH₃)₂, cyano (—CN), sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, —OCF₃, C₁-C₁₀ alkyl, C₂-C₁₀ alkenyl, C₂-C₁₀ alkynyl, or C₁-C₉ alkoxy, which methanoyl (—COH), —NH₂, —N(CH₃)₂, —CONH₂, —CON(CH₃)₂, —C(O)(CH₃), —C(O)(C₂H₅), —C(O)(C₃H₇), C₁-C₁₀ alkyl, C₂-C₁₀ alkenyl, C₂-C₁₀ alkynyl, C₁-C₉ alkoxy can optionally be substituted with one or more of halogen, oxo (=O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluoronated alkyl, —CF₃, —OCF₃, or C₁-C₃ alkoxy;
n1 can be 1, 2, 3, 4, 5, 6, 7, 8, or 9;
n2 can be 0, 1, 2, 3, or 4;
n3 can be 0, 1, 2, 3, or 4;
Y can be —CH₂—, —O—(CO)—, —O—(N═CH)—, —O—, or —N(R^{g})—(CO)—; (Of course, these moieties can be reversed; for example, —O—(CO)— represents both —O—(CO)— and —(CO)—O—)
Z can be —CH₂—, —O—(CO)—, —O—(N═CH)—, —O—, or —N(R^{h})—(CO)—; (Of course, these moieties can be reversed; for example, —O—(CO)— represents both —O—(CO)— and —(CO)—O—)
R^{g} and R^{h} can be the same or different and are H, methyl, ethyl, propyl, or butyl; and
R³ and R⁴ can be the same or different and are C₆-C₃₀ alkyl, C₆-C₃₀ alkenyl, C₆-C₃₀ alkynyl.

2. The boron compound of embodiment 1, wherein X is —C(R^{a})(R^{b})— and/or n1 is 1 and/or n2 is 0, and/or n3 is 1 and/or Y can be —O—(CO)— and/or Z can be —O—(CO)—.

3. The boron compound of embodiment 1 or embodiment 2, wherein R^{a}, R^{b}, R^{c}, R^{d}, R^{e}, and R^{f} are H.

4. The boron compound of any of embodiments 1-3, wherein R¹ and R² are methyl or ethyl.

5. The boron compound of any of embodiments 1-4, wherein R³ and R⁴ are the same and are C₁₃ alkyl.

6. The boron compound of any of embodiments 1-4, wherein R³ and R⁴ are the same and are C₁₇ alkenyl (A9).

7. The boron compound of any of embodiments 1-6, wherein the pKₐ of the boron compound is 5-8 or 6-7.

8. The boron compound of any of embodiments 1-7, wherein the boron compound is DMDBH or DODBH.

9. A lipid particle (e.g., a lipid nanoparticle or a liposome or a liposome formulation) comprising the boron compound of any of embodiments 1-8.

10. The lipid particle of embodiment 9, wherein the amount of the boron compound in the lipid particle is 0.01 to 50 mol % or 0.1 to 25 mol % or 1 to 10 mol %.

11. The lipid particle of any of embodiments 9-10, wherein (a) the boron compound reacts with a lipid particle component (e.g., a molecule with a diol, a molecule with a 1,2 diol, a molecule with a 1,3 diol, a sugar, an amino acid, a peptide, a polypeptide, a polynucleotide, a PEG polymer, and the like) in the lipid particle to form one or more covalent bonds between the boron compound and the lipid particle component, and (b) the lipid particle component is not a boron compound.

12. The lipid particle of any of embodiments 9-11, wherein the lipid particle is a molecule with a diol (e.g., a molecule with a 1,2 diol or a molecule with a 1,3 diol).

13. The lipid particle of any of embodiments 9-12, wherein the lipid particle component is OEL4.

14. The lipid particle of any of embodiments 9-13, wherein the diameter of the lipid particle is decreased by at least 10%, by at least 20%, by at least 30%, by at least 40%, by at least 50%, by at least 60%, by at least 70%, or by at least 80%, compared to the lipid particle without addition of the boron compound.

15. The lipid particle of any of embodiments 9-14, wherein the diameter of the lipid particle is 30-250 nm, 50-110 nm, 60-105 nm, or 65-100 nm.

16. A lipoplex comprising (a) the boron compound of any of embodiments 1-8 or the lipid particle of any of embodiments 9-15 and (b) a nucleic acid molecule (e.g., ssDNA, dsDNA, ssRNA dsRNA, or mRNA), such as a nucleic acid molecule having a molecular weight (in daltons) of no more than 10,000,000, no more than 2,000,000, no more than 1,000,000, no more than 500,000, no more than 100,000, or no more than 10,000.

17. A composition comprising the boron compound of any of embodiments 1-8, the lipid particle of any of embodiments 9-15, or the lipoplex of embodiment 16.

18. A pharmaceutical composition comprising (a) the boron compound of any of embodiments 1-8, the lipid particle of any of embodiments 9-15, or the lipoplex of embodiment 16 and (b) optionally a pharmaceutically acceptable excipient.

19. A method for silencing a gene in a cell (e.g., animal cell, mammalian cell, or human cell) comprising one or more administrations to the cell of one or more compositions comprising the lipoplex of embodiment 16, wherein the compositions may be the same or different if there is more than one administration.

20. A method of embodiment 19, wherein the cell is in vivo, ex vivo, or in vitro.

21. A method for providing an animal with a compound comprising one or more administrations to the animal of one or more compositions comprising the lipoplex of embodiment 16, wherein the compositions may be the same or different if there is more than one administration.

22. The method of embodiment 21, wherein at least one of the one or more compositions further comprises a formulary ingredient.

23. The method of embodiment 21 or embodiment 22, wherein at least one of the one or more compositions comprises the composition of embodiment 17 or the pharmaceutical composition of embodiment 18.

24. The method of any of embodiments 21-23, wherein at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration.

25. The method of any of embodiments 21-24, wherein if there is more than one administration at least one composition used for at least one administration is different from the composition of at least one other administration.

26. The method of any of embodiments 21-25, wherein the compound of at least one of the one or more compositions is administered to the animal in an amount of from about 0.01 mg/kg animal body weight to about 15 mg/kg animal body weight.

27. The method of any of embodiments 21-26, wherein the animal is a human, a rodent, or a primate.

28. A method for treating an animal for a disease, comprising one or more administrations of one or more compositions comprising the lipoplex of embodiment 16, wherein the compositions may be the same or different if there is more than one administration.

29. The method of embodiment 28, wherein at least one of the one or more compositions further comprises a formulary ingredient.

30. The method of embodiment 28 or embodiment 29, wherein at least one of the one or more compositions comprises the composition of embodiment 17 or the pharmaceutical composition of embodiment 18.

31. The method of any of embodiments 28-30, wherein at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration.

32. The method of any of embodiments 28-31, wherein if there is more than one administration at least one composition used for at least one administration is different from the composition of at least one other administration.

33. The method of any of embodiments 28-32, wherein the compound of at least one of the one or more compositions is administered to the animal in an amount of from about 0.005 mg/kg animal body weight to about 50 mg/kg animal body weight.

34. The method of any of embodiments 28-33, wherein the animal is a human, a rodent, or a primate.

35. The method of any of embodiments 28-34, wherein the animal is in need of the treatment.

36. The method of any of embodiments 28-35, wherein the method is for treating cancer.

37. The method of any of embodiments 28-36, wherein the method is for treating acute lymphoblastic leukemia, astrocytoma, basal cell carcinoma, bladder cancer, bone marrow cancer, breast cancer, chronic lymphocytic leukemia (CLL), CNS cancer, colon cancer, colorectal cancer, endometrial cancer, gastric cancer, glioblastoma, glioblastoma multiforme, glioma, gliosarcoma, hepatocellular carcinoma, kidney cancer, leukemia, liver cancer, lung cancer, lymphoma, melanoma, malignant nerve sheath tumors, medulloblastoma, meningioma, multiple myeloma, nasopharyngeal carcinoma, neuroblastoma, non-Hodgkin lymphoma, non-small cell lung cancer, oral cancer, ovarian cancer, pancreatic cancer, prostate cancer, rectal cancer, renal cancer, renal cell carcinoma, rhabdomyosarcoma, squamous cell carcinoma, stomach cancer, thyroid cancer, uterine cancer, cancers that can result in metastasis, cancers resulting from metastasis, or cancerous tumors thereof.

38. The method of any of embodiments 28-37, wherein the method is for treating cancerous tumors.

39. A method for preparing the boron compound of any of embodiments 1-8 using any suitable method, such as those disclosed herein.

40. A method for preparing the lipid particle of any of embodiments 9-15, using any suitable method, such as those disclosed herein.

41. A method for preparing the lipoplex of embodiment 16 using any suitable method, such as those disclosed herein.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A boron compound selected from Formula (I), salts of Formula (I), optical isomers of Formula (I), geometric isomers of Formula (I), salts of optical isomers of Formula (I), salts of geometric isomers of Formula (I), and derivatives thereof, where Formula (I) is Formula (Ia) and Formula (Ib), 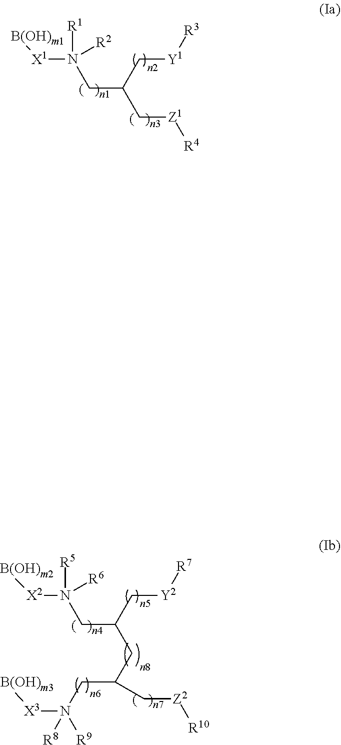
wherein
X, X², and X³ can be the same or different and are —C(R^{a})(R^{b})—, —C(R^{a})(R^{b})—C(R^{c})(R^{d})—, or —C(R^{a})(R^{b})—C(R^{c})(R^{d})—C(R^{e})(R^{f})—, where R^{a}, R^{b}, R^{c}, R^{d}, R^{e}, and R^{f} can be the same or different if they are on different X groups;
R^{a}, R^{b}, R^{c}, R^{d}, R^{e}, and R^{f} can be the same or different and are H, halogen (e.g., F, Cl, Br, or I), —CN, hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, or —OCF₃;
R¹, R², R⁵, R⁶, R⁸, and R⁹ can be the same or different and are a lone pair of electrons, H, hydroxy (—OH), methanoyl (—COH), nitro (—NO₂), —NH₂, —N(CH₃)₂, cyano (—CN), sulfo (—SO₃H), —SO₂(aryl), —SO₂(alkyl), —CONH₂, —CON(CH₃)₂, —C(O)(CH₃), —C(O)(C₂H₅), —C(O)(C₃H₇), C₁-C₃ perfluoronated alkyl, —CF₃, —OCF₃, C₁-C₁₀ alkyl, C₂-C₁₀ alkenyl, C₂-C₁₀ alkynyl, or C₁-C₉ alkoxy, which methanoyl (—COH), —NH₂, —N(CH₃)₂, —CONH₂, —CON(CH₃)₂, —C(O)(CH₃), —C(O)(C₂H₅), —C(O)(C₃H₇), C₁-C₁₀ alkyl, C₂-C₁₀ alkenyl, C₂-C₁₀ alkynyl, C₁-C₉ alkoxy can optionally be substituted with one or more of halogen, oxo (=O), hydroxy (—OH), methanoyl (—COH), carboxy (—CO₂H), nitro (—NO₂), —NH₂, —N(CH₃)₂, cyano (—CN), ethynyl (—CCH), propynyl, sulfo (—SO₃H), morpholinyl, —CO-morpholin-4-yl, phenyl, —CONH₂, —CON(CH₃)₂, C₁-C₃ alkyl, C₁-C₃ perfluoronated alkyl, —CF₃, —OCF₃, or C₁-C₃ alkoxy;
m1 is 2 or 3;
m2 is 2 or 3;
m3 is 2 or 3;
n1 is 1, 2, 3, 4, 5, 6, 7, 8, or 9;
n2 is 0, 1, 2, 3, or 4;
n3 is 0, 1, 2, 3, or 4;
n4 is 1, 2, 3, 4, 5, 6, 7, 8, or 9;
n5 is 0, 1, 2, 3, or 4;
n6 is 0, 1, 2, 3, 4, 5, 6, 7, 8, or 9;
n7 is 0, 1, 2, 3, or 4;
n8 is 0, 1, 2, or 3;
Y¹ and Y² can be the same or different and are —CH₂—, —O—(CO)—, —(CO)—O—, —O—(N═CH)—, —(CH═N)—O—, —O—, —N(R^{g})—(CO)—, or —(CO)—N(R^{g})—, where R^{g} can be the same or different if they are on different Y groups;
Z¹ and Z² can be the same or different and are —CH₂—, —O—(CO)—, —(CO)—O—, —O—(N═CH)—, —(CH═N)—O—, —O—, —N(R^{h})—(CO)—, or —(CO)—N(R^{h})—, where R^{h} can be the same or different if they are on different Z groups;
R^{g} and R^{h} can be the same or different and are H, methyl, ethyl, propyl, or butyl; and
R³, R⁴, R⁷ and R¹⁰ can be the same or different and are C₆-C₃₀ alkyl, C₆-C₃₀ alkenyl, or C₆-C₃₀ alkynyl.

2. The boron compound of claim 1, wherein Formula (I) is 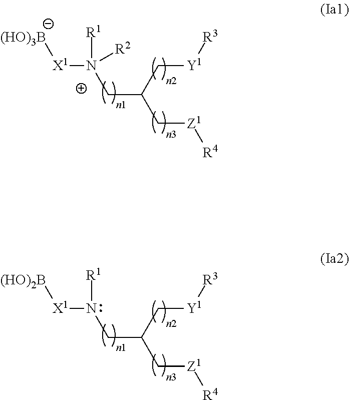
wherein R¹ is not a lone pair of electrons in Formula (Ia2).

3. The boron compound of claim 1 or claim 2, wherein X¹, X², and X³ is —C(R^{a})(R^{h})—.

4. The boron compound of any of the preceding claims, wherein R^{a}, R^{b}, R^{c}, R^{d}, R^{e}, and R^{f} are H.

5. The boron compound of any of the preceding claims, wherein R¹ and R² can be the same or different and are H, methyl, or ethyl.

6. The boron compound of any of the preceding claims, wherein R⁵ and R⁶ can be the same or different and are H, methyl, or ethyl.

7. The boron compound of any of the preceding claims, wherein R⁸ and R⁹ can be the same or different and are H, methyl, or ethyl.

8. The boron compound of any of the preceding claims, wherein R³ and R⁴ are the same or different and are C₁₂-C₁₈ alkyl or C₁₂-C₁₈ alkenyl.

9. The boron compound of any of the preceding claims, wherein R⁷ and R¹⁰ are the same or different and are C₁₂-C₁₈ alkyl or C₁₂-C₁₈ alkenyl.

10. The boron compound of any of the preceding claims, wherein Y¹ and Y² are the same or different and are —O—(CO)—, —O—(N═CH)—, or —O—.

11. The boron compound of any of the preceding claims, wherein Z¹ and Z² are the same or different and are —O—(CO)—, —O—(N═CH)—, or —O—.

12. The boron compound of any of the preceding claims, wherein X¹ is —CH₂—, R¹ is —CH₃, R² is —CH₃, n1 is 1, n2 is 0, n3 is 1, Y¹ is —O—(CO)—, or Z¹ is —O—(CO)—, or a combination thereof.

13. The boron compound of any of the preceding claims, wherein X² is —CH₂—, X³ is —CH₂—, R⁵ is —CH₃, R⁶ is —CH₃, R⁸ is —CH₃, R⁹ is —CH₃, n4 is 1, n6 is 0, n5 is 0, n7 is 0, n8 is 0, Y² is —O—(CO)—, or Z² is —O—(CO)—, or a combination thereof.

14. The boron compound of any of the preceding claims, wherein a pKₐ of the boron compound is 5 to 8 or 6 to 7.

15. The boron compound of any of the preceding claims, wherein the boron compound is I-1, I-2, I-3, I-4, I-5, I-6, I-7, I-8, I-9 I-10, I-11, I-12, I-13, I-14, I-15, I-16, or I-17.

16. The boron compound of any of the preceding claims, wherein the boron compound is I-1, I-2, or I-3.

17. A lipid particle comprising the boron compound of any of claims 1-16.

18. The lipid particle of claim 17, wherein the amount of the boron compound in the lipid particle is 0.01 to 50 mol %.

19. The lipid particle of any of claims 17-18, wherein (a) the boron compound reacts with a lipid particle component in the lipid particle to form one or more covalent bonds between the boron compound and the lipid particle component, and (b) the lipid particle component is not a boron compound.

20. The lipid particle of any of claims 17-19, wherein the lipid particle component is a molecule with a diol, a molecule with a 1,2 diol, a molecule with a 1,3 diol, or OEL4.

21. The lipid particle of any of claims 17-20, wherein the diameter of the lipid particle is decreased by at least 10%, compared to the lipid particle without addition of the boron compound.

22. The lipid particle of any of claims 17-21, wherein the diameter of the lipid particle is 30-250 nm.

23. A lipoplex comprising (a) the boron compound of any of claims 1-16 or the lipid particle of any of claims 17-22 and (b) a nucleic acid molecule.

24. The lipoplex of claim 23, wherein the nucleic acid molecule has a molecular weight (in daltons) of no more than 10,000,000.

25. A composition comprising the boron compound of any of claims 1-16, the lipid particle of any of claims 17-22, or the lipoplex of any of claims 23-24.

26. A pharmaceutical composition comprising (a) the boron compound of any of claims 1-16, the lipid particle of any of claims 17-22, or the lipoplex of any of claims 23-24 and (b) optionally a formulary ingredient.

27. A method for providing nucleic acid to a cell (e.g., plant, cell, animal cell, mammalian cell, or human cell) comprising one or more administrations to the cell of one or more compositions comprising the lipoplex of claim 23-24, the composition of claim 25, or the pharmaceutical composition of claim 26, wherein the compositions may be the same or different if there is more than one administration.

28. The method of claim 27, wherein the method silences a gene in the cell or the added nucleic acid results in the expression of a protein or a polypeptide in the cell.

29. The method of claim 27 or claim 28, wherein the cell is in vivo, ex vivo, or in vitro.

30. A method for providing an animal with a compound comprising one or more administrations to the animal of one or more compositions comprising the lipoplex of claim 23-24, the composition of claim 25, or the pharmaceutical composition of claim 26, wherein the compositions may be the same or different if there is more than one administration.

31. The method of claim 30, wherein at least one of the one or more compositions further comprises a formulary ingredient.

32. The method of claim 30 or claim 31, wherein at least one of the one or more compositions comprises the composition of claim 25 or the pharmaceutical composition of claim 26.

33. The method of any of claims 30-32, wherein at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration.

34. The method of any of claims 30-33, wherein if there is more than one administration at least one composition used for at least one administration is different from the composition of at least one other administration.

35. The method of any of claims 30-34, wherein the lipoplex of at least one of the one or more compositions is administered to the animal in an amount of from about 0.01 mg/kg animal body weight to about 15 mg/kg animal body weight.

36. The method of any of claims 30-35, wherein the animal is a human, a rodent, or a primate.

37. A method for treating an animal for a disease, comprising one or more administrations of one or more compositions comprising the lipoplex of claim 23-24, the composition of claim 25, or the pharmaceutical composition of claim 26, wherein the compositions may be the same or different if there is more than one administration.

38. The method of claim 37, wherein at least one of the one or more compositions further comprises a formulary ingredient.

39. The method of claim 37 or claim 38, wherein at least one of the one or more compositions comprises the composition of claim 25 or the pharmaceutical composition of claim 26.

40. The method of any of claims 37-39, wherein at least one of the one or more administrations comprises parenteral administration, a mucosal administration, intravenous administration, subcutaneous administration, topical administration, intradermal administration, oral administration, sublingual administration, intranasal administration, intrathecal administration, or intramuscular administration.

41. The method of any of claims 37-40, wherein if there is more than one administration at least one composition used for at least one administration is different from the composition of at least one other administration.

42. The method of any of claims 37-41, wherein the lipoplex of at least one of the one or more compositions is administered to the animal in an amount of from about 0.005 mg/kg animal body weight to about 50 mg/kg animal body weight.

43. The method of any of claims 37-42, wherein the animal is a human, a rodent, or a primate.

44. The method of any of claims 37-43, wherein the animal is in need of the treatment.

45. The method of any of claims 37-44, wherein the method is for treating cancer.

46. The method of any of claims 37-45, wherein the method is for treating acute lymphoblastic leukemia, astrocytoma, basal cell carcinoma, bladder cancer, bone marrow cancer, breast cancer, chronic lymphocytic leukemia (CLL), CNS cancer, colon cancer, colorectal cancer, endometrial cancer, gastric cancer, glioblastoma, glioblastoma multiforme, glioma, gliosarcoma, hepatocellular carcinoma, kidney cancer, leukemia, liver cancer, lung cancer, lymphoma, melanoma, malignant nerve sheath tumors, medulloblastoma, meningioma, multiple myeloma, nasopharyngeal carcinoma, neuroblastoma, non-Hodgkin lymphoma, non-small cell lung cancer, oral cancer, ovarian cancer, pancreatic cancer, prostate cancer, rectal cancer, renal cancer, renal cell carcinoma, rhabdomyosarcoma, squamous cell carcinoma, stomach cancer, thyroid cancer, uterine cancer, cancers that can result in metastasis, cancers resulting from metastasis, or cancerous tumors thereof.

47. The method of any of claims 37-46, wherein the method is for treating cancerous tumors.

48. The method of any of claims 37-47, wherein the method comprises a vaccination.

49. The method of any of claims 37-48, wherein the method is for treating infections.

50. The method of any of claims 37-49, wherein the method is for treating bacterial infections, viral infections, fungal infections, or a combination thereof.

51. The method of any of claims 27-50, wherein the method induces an immune response.

52. A method for preparing the boron compound of Formula (Ia) and salts thereof of any of claims 1-16, comprising:
(a) reacting a compound of Formula (IIa) with R³—(O)C-(halogen) and R⁴—(O)C-(halogen) to result in a mixture comprising a compound of Formula (IIIa);
(b) reacting a compound of Formula (IIIa) with 2-(halomethyl)-4,4,5,5-tetramethyl-1,3,2-dioxaborolane to result in a mixture comprising a compound of Formula (IVa);
(c) reacting a compound of Formula (IVa) with any suitable molecule to oxidatively create B—OH groups; and
(d) recovering Formula (Ia) or a salt thereof,
where Formula (IIa) is 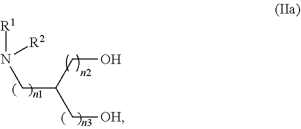
Formula (IIIa) is 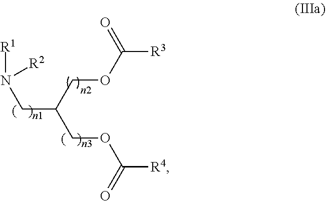
and
Formula (IVa) is

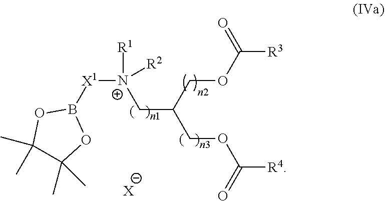

53. A method for preparing the boron compound of Formula (Ib) and salts thereof of any of claims 1-16, comprising:
(a) reacting a compound of Formula (IIb) with R⁷—(O)C-(halogen) and R¹⁰—(O)C-(halogen) to result in a mixture comprising a compound of Formula (IIIb);
(b) reacting a compound of Formula (IIIb) with 2-(halomethyl)-4,4,5,5-tetramethyl-1,3,2-dioxaborolane pinacol ester to result in a mixture comprising a compound of Formula (IVb);
(c) reacting a compound of Formula (IVb) with any suitable molecule to oxidatively create B—OH groups; and
(d) recovering Formula (Ib) or a salt thereof
where Formula (IIb) is 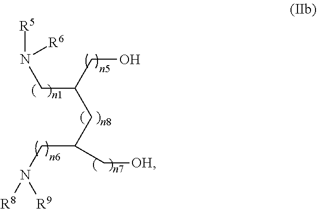
Formula (IIIb) is 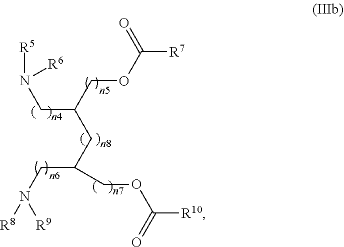
and
Formula (IVb) is [chemical structure]


SEQUENCE LISTING (2 sequences)

SEQ ID NO: 1 - RNA, 25 nt, synthetic construct
  source 1..25: mol_type = other RNA
accctgaagttcatctgcaccaccg

SEQ ID NO: 2 - RNA, 27 nt, synthetic construct
  source 1..27: mol_type = other RNA
cggtggtgcagatgaacttcagggtca